(19) United States
(12) Patent Application Publication
(10) Pub. No.: US 2025/0339449 A1
(43) Pub. Date: Nov. 6, 2025

(54) INHALATION FORMULATIONS CONTAINING NITRIC OXIDE RELEASING COMPOUNDS AND METHODS OF USING SAME

(71) Applicant: KNOW Bio, LLC, Morrisville, NC (US)

(72) Inventors: Nathan Allan Stasko, Chapel Hill, NC (US); John Kelly Simons, Cary, NC (US)

(73) Assignee: KNOW Bio, LLC, Morrisville, NC (US)

(21) Appl. No.: 19/197,332

(22) Filed: May 2, 2025

Related U.S. Application Data
(60) Provisional application No. 63/641,680, filed on May 2, 2024.

(51) Int. Cl. A61K 31/655; A61K 9/00; A61K 45/06

(52) U.S. Cl. CPC A61K 31/655; A61K 9/0075; A61K 45/06

(57) ABSTRACT

Provided herein are formulations designed for administration by inhalation, including aqueous based compositions and dry powder compositions. The compositions described herein comprise a nitric oxide (NO) releasing compound, along with one or more additional agents, additives, and/or carriers. The compositions described herein exhibit a heat of decomposition of no greater than 300 J/g. Also described herein are methods of treating respiratory diseases in a subject, comprising administering to the subject an effective amount of a composition described herein.

### CROSS REFERENCE TO PRIORITY APPLICATION

The application claims priority to U.S. Provisional Application No. 63/641,680, filed May 2, 2024, which is incorporated herein by reference in its entirety.

### FIELD

The present disclosure relates to formulations suitable for administration by inhalation, and includes formulations prepared from aqueous based compositions and dry powder compositions. The present disclosure also relates to methods of using the formulations for treating one or more respiratory diseases in a subject.

### BACKGROUND

Many respiratory diseases can be caused by bacteria and other microbes, in which the microbes infect the lungs, throat, and airways of the respiratory system. Nitric oxide has many therapeutic uses, including use as an antimicrobial agent. Nitric oxide gas can be administered to subjects via inhalation; however, such administration is difficult, time-consuming, and potentially toxic to the individual based on the required amount and duration of exposure necessary. Nitric-oxide releasing compounds have also been explored as therapeutic agents for delivering nitric oxide. Such compounds must be formulated, however, to ensure adequate stability prior to and during administration by the subject and subsequently adequate levels of nitric oxide delivered to the lung at a desirable release rate, and in a manner that encourages patient compliance.

### SUMMARY

Described herein are formulations designed for administration by inhalation, including formulations prepared from aqueous based compositions and dry powder compositions. Described herein is a composition comprising a nitric oxide (NO) releasing compound and an aqueous carrier, wherein the composition exhibits a heat of decomposition of no greater than 300 J/g. The composition can be formulated for inhalation. Optionally, the composition further comprises a pharmaceutically acceptable carrier. In some cases, the composition further comprises one or more additives (e.g., one or more of carbohydrates, organic salts, amino acids, preservatives, salts, chelators, viscosity modifiers, stabilizers, surfactants, antioxidants, polymers, mucolytics, glycols, or cosolvents). In some cases, the composition further comprises an additional active agent, such as a peptide, a protein, a corticosteroid, an antibiotic, a short acting beta agonist, a long acting beta agonist, an anti-cholinergic, an antiviral, a monoclonal antibody (mAb), and/or an antifungal.

Optionally, the nitric oxide (NO) releasing compound comprises at least one of a diazeniumdiolate (e.g., an N-diazeniumdiolate or a C-diazeniumdiolate), a nitrosothiol, a nitrosourea, an organic nitrate, an organic nitrite, a metal-NO complex, an N-nitroimine, a C-nitroso compound, an oxime, an N-hydroxyguanidine, or a hydroxyurea. In some cases, the nitric oxide (NO) releasing compound comprises at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

The nitric oxide (NO) releasing compound can have the following structure:

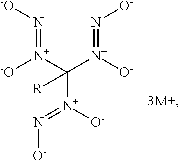

wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H; a, b, c, d, e, and f are each independently selected from an integer of 0, 1, 2, 3, 4, 5, 6, 7, 8, 9, or 10; and M+ is a pharmaceutically-acceptable cation, wherein a ratio of the compound to the cation is such that the overall net charge of the compound is neutral. The cation can optionally be selected from the group consisting of sodium, potassium, lithium, calcium, magnesium, ammonium, and substituted ammonium. In some examples, the compound has the following structure:

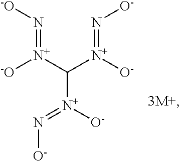

such as

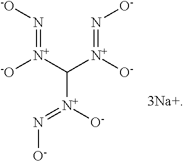

Also described herein is a method for treating a respiratory disease in a subject, comprising administering to the subject an effective amount of a composition as described herein. The composition can be contained in a drug administration device. Optionally, the drug administration device comprises a container attached to a pump. The administering can comprise aerosolizing the composition. Optionally, the drug administration device is at least one of a spray and a soft mist inhaler.

Also described herein is a composition comprising a nitric oxide (NO) releasing compound and a propellant, wherein the composition exhibits a heat of decomposition of no greater than 300 J/g. Optionally, the composition is formulated for administration by an inhaler, such as a metered dose inhaler. In some cases, the propellant is a fluorinated hydrocarbon (e.g., 1,1,1,2-tetrafluoroethane, 1,1,1,2,3,3,3-heptafluoropropane, trans-1,3,3,3-tetrafluoropropene, 1,1-difluoroethane, or a combination thereof).

In some cases, the composition further comprises one or more additives (e.g., one or more of carbohydrates, organic salts, amino acids, preservatives, salts, chelators, viscosity modifiers, stabilizers, surfactants, antioxidants, polymers, mucolytics, glycols, or cosolvents). In some cases, the composition further comprises an additional active agent, such as a peptide, a protein, a corticosteroid, an antibiotic, a short acting beta agonist, a long acting beta agonist, an anti-cholinergic, an antiviral, a monoclonal antibody (mAb), and/or an antifungal. Optionally, the composition further comprises ethanol.

Optionally, the nitric oxide (NO) releasing compound comprises at least one of a diazeniumdiolate (e.g., an N-diazeniumdiolate or a C-diazeniumdiolate), a nitrosothiol, a nitrosourea, an organic nitrate, an organic nitrite, a metal-NO complex, an N-nitroimine, a C-nitroso compound, an oxime, an N-hydroxyguanidine, or a hydroxyurea. In some cases, the nitric oxide (NO) releasing compound comprises at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

The nitric oxide (NO) releasing compound can have the following structure:

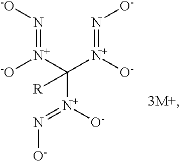

wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H; a, b, c, d, e, and f are each independently selected from an integer of 0, 1, 2, 3, 4, 5, 6, 7, 8, 9, or 10; and M+ is a pharmaceutically-acceptable cation, wherein a ratio of the compound to the cation is such that the overall net charge of the compound is neutral. The cation can optionally be selected from the group consisting of sodium, potassium, lithium, calcium, magnesium, ammonium, and substituted ammonium. In some examples, the compound has the following structure:

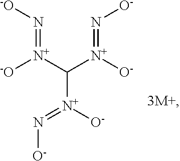

such as

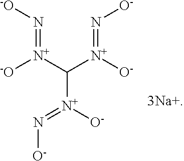

Also described herein is a method for treating a respiratory disease in a subject, comprising administering to the subject an effective amount of a composition as described herein. The composition can be contained in a drug administration device. Optionally, the drug administration device is a propellant administration device. The administering can comprise aerosolizing the composition. Optionally, the drug administration device is an inhaler (e.g., a metered dose inhaler).

Further described herein is a composition comprising a nitric oxide (NO) releasing compound and an aqueous based vaporizable liquid, wherein the composition exhibits a heat of decomposition of no greater than 300 J/g. The aqueous based vaporizable liquid can comprise water and one or more of propylene glycol and vegetable glycerine. Optionally, the composition is formulated for administration by an electronic vaporizing device.

In some cases, the composition further comprises one or more additives (e.g., one or more of carbohydrates, organic salts, amino acids, preservatives, salts, chelators, viscosity modifiers, stabilizers, surfactants, antioxidants, polymers, mucolytics, glycols, or cosolvents). In some cases, the composition further comprises an additional active agent, such as a peptide, a protein, a corticosteroid, an antibiotic, a short acting beta agonist, a long acting beta agonist, an anti-cholinergic, an antiviral, a monoclonal antibody (mAb), and/or an antifungal.

Optionally, the nitric oxide (NO) releasing compound comprises at least one of a diazeniumdiolate (e.g., an N-diazeniumdiolate or a C-diazeniumdiolate), a nitrosothiol, a nitrosourea, an organic nitrate, an organic nitrite, a metal-NO complex, an N-nitroimine, a C-nitroso compound, an oxime, an N-hydroxyguanidine, or a hydroxyurea. In some cases, the nitric oxide (NO) releasing compound comprises at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

The nitric oxide (NO) releasing compound can have the following structure:

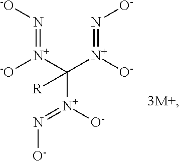

wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H; a, b, c, d, e, and f are each independently selected from an integer of 0, 1, 2, 3, 4, 5, 6, 7, 8, 9, or 10; and M+ is a pharmaceutically-acceptable cation, wherein a ratio of the compound to the cation is such that the overall net charge of the compound is neutral. The cation can optionally be selected from the group consisting of sodium, potassium, lithium, calcium, magnesium, ammonium, and substituted ammonium. In some examples, the compound has the following structure:

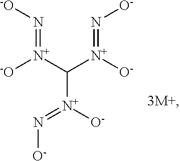

such as

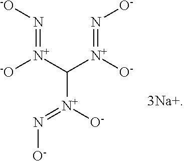

Also described herein is a method for treating a respiratory disease in a subject, comprising administering to the subject an effective amount of a composition as described herein. The composition can be contained in a drug administration device. Optionally, the drug administration device is an electronic vaporizing device. The administering can comprise vaporizing the composition.

Further described herein is a composition comprising a nitric oxide (NO) releasing compound and a carrier particle, wherein the composition exhibits a heat of decomposition of no greater than 300 J/g. Optionally, the composition is formulated for inhalation. The carrier particle can comprise at least one of lactose and a stearate treated lactose. Optionally, the carrier particle comprises a stearate treated lactose and the stearate treated lactose comprises a metal cationic salt selected from the group consisting of a magnesium salt, a potassium salt, a sodium salt, or a calcium salt.

In some cases, the composition further comprises one or more additives (e.g., one or more of carbohydrates, organic salts, amino acids, preservatives, salts, chelators, viscosity modifiers, stabilizers, surfactants, antioxidants, polymers, mucolytics, glycols, or cosolvents). In some cases, the composition further comprises an additional active agent, such as a peptide, a protein, a corticosteroid, an antibiotic, a short acting beta agonist, a long acting beta agonist, an anti-cholinergic, an antiviral, a monoclonal antibody (mAb), and/or an antifungal.

Optionally, the nitric oxide (NO) releasing compound comprises at least one of a diazeniumdiolate (e.g., an N-diazeniumdiolate or a C-diazeniumdiolate), a nitrosothiol, a nitrosourea, an organic nitrate, an organic nitrite, a metal-NO complex, an N-nitroimine, a C-nitroso compound, an oxime, an N-hydroxyguanidine, or a hydroxyurea. In some cases, the nitric oxide (NO) releasing compound comprises at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

The nitric oxide (NO) releasing compound can have the following structure:

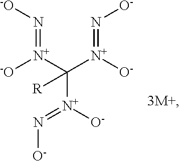

wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H; a, b, c, d, e, and f are each independently selected from an integer of 0, 1, 2, 3, 4, 5, 6, 7, 8, 9, or 10; and M₊ is a pharmaceutically-acceptable cation, wherein a ratio of the compound to the cation is such that the overall net charge of the compound is neutral. The cation can optionally be selected from the group consisting of sodium, potassium, lithium, calcium, magnesium, ammonium, and substituted ammonium. In some examples, the compound has the following structure:

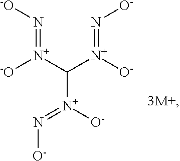

such as

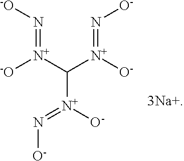

Also described herein is a method for treating a respiratory disease in a subject, comprising administering to the subject an effective amount of a composition as described herein. The composition can be contained in a drug administration device. Optionally, the drug administration device is a dry powder inhaler. The administering can comprise aerosolizing the composition.

Further described herein is a composition comprising a porous particle comprising a nitric oxide (NO) releasing compound, wherein the composition exhibits a heat of decomposition of no greater than 300 J/g. Optionally, the composition is formulated for administration by an inhaler (e.g., a dry powder inhaler). In some cases, a mass median aerodynamic diameter of the porous particle is no greater than 10 microns in diameter (e.g., no greater than 5 microns in diameter or no greater than 3 microns in diameter).

In some cases, the composition further comprises one or more additives (e.g., one or more of carbohydrates, organic salts, amino acids, preservatives, salts, chelators, viscosity modifiers, stabilizers, surfactants, antioxidants, polymers, mucolytics, glycols, or cosolvents). In some cases, the composition further comprises an additional active agent, such as a peptide, a protein, a corticosteroid, an antibiotic, a short acting beta agonist, a long acting beta agonist, an anti-cholinergic, an antiviral, a monoclonal antibody (mAb), and/or an antifungal.

Optionally, the nitric oxide (NO) releasing compound comprises at least one of a diazeniumdiolate (e.g., an N-diazeniumdiolate or a C-diazeniumdiolate), a nitrosothiol, a nitrosourea, an organic nitrate, an organic nitrite, a metal-NO complex, an N-nitroimine, a C-nitroso compound, an oxime, an N-hydroxyguanidine, or a hydroxyurea. In some cases, the nitric oxide (NO) releasing compound comprises at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

The nitric oxide (NO) releasing compound can have the following structure:

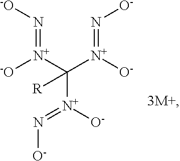

wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H; a, b, c, d, e, and f are each independently selected from an integer of 0, 1, 2, 3, 4, 5, 6, 7, 8, 9, or 10; and M₊ is a pharmaceutically-acceptable cation, wherein a ratio of the compound to the cation is such that the overall net charge of the compound is neutral. The cation can optionally be selected from the group consisting of sodium, potassium, lithium, calcium, magnesium, ammonium, and substituted ammonium. In some examples, the compound has the following structure:

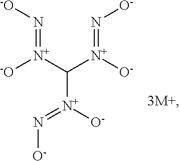

such as

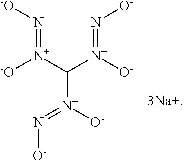

Also described herein is a method for treating a respiratory disease in a subject, comprising administering to the subject an effective amount of a composition as described herein. The composition can be contained in a drug administration device. Optionally, the drug administration device is a dry powder inhaler.

The details of one or more embodiments are forth in the description below. Other features, objects, and advantages will be apparent from the description and from the claims.

### DETAILED DESCRIPTION

Described herein are formulations designed for administration by inhalation, including formulations prepared from aqueous based compositions and dry powder compositions. The compositions can be formulated for inhalation and include a nitric oxide (NO) releasing compound and optionally, one or more excipients and/or carriers. The excipients and carriers included in the compositions are tailored to the desired form of administration. Optionally, the compositions further include one or more additives and/or one or more additional active agents.

The compositions advantageously exhibit a heat of decomposition of no greater than 300 J/g (e.g., less than 275 J/g, less than 250 J/g, less than 225 J/g, less than 200 J/g, less than 175 J/g, less than 150 J/g, less than 125 J/g, less than 100 J/g, less than 75 J/g, or less than 50 J/g). As known to those of ordinary skill in the art, a substance is considered a candidate for classification as a United Nations Class 1 explosive if the heat of decomposition is ≥500 J/g. While a substance with a higher energy content is not necessarily highly sensitive or hazardous, the sensitivity of a high energy content material must be understood to ensure safe handling. Nitric oxide (NO) releasing compound MD3 (Compound 1 herein) undergoes violent exothermic decomposition (detonation) in the solid state at 187° C. As outlined below in Example 1, the decomposition energy of MD3 is 3754 J/g. The novel compositions described herein, however, address the thermal decomposition nature of nitric oxide (NO) releasing compounds, resulting in therapeutically effective formulations that can be safely stored and handled.

### I. Compositions

As described above, the compositions for use in the formulations described herein include a nitric oxide (NO) releasing agent, optionally in combination with one or more additional active agents and/or one or more additives.

a. Nitric Oxide (NO) Releasing Agent

An active pharmaceutical ingredient delivered via the first component described herein is nitric oxide and can be included in the first component in the form of a compound that releases nitric oxide (NO) (e.g., nitric oxide donors, nitric oxide releasing prodrugs, or compounds necessary to facilitate the endogenous generation of nitric oxide). As described herein, the first component in the form of a compound that releases NO can exhibit antimicrobial characteristics, including antiviral, antibacterial, and antifungal characteristics. The firm component described herein can further possess anti-inflammatory properties and other beneficial therapeutic properties.

The NO release can be initiated thermally or via any of the degradation strategies for the labile portion of N-diazeniumdiolates, nitrosamines, hydroxyl nitrosamines, nitrosothiols, hydroxylamines, hydroxyureas, metal complexes, organic nitrites and organic nitrates. See, Wang, P. G., et al., Nitric Oxide Donors. For Pharmaceutical and Biological Applications; Wliey-VCH. Weinheim, Germany, 2005; and Wang. P. G., et al., Chem. Rev., 102, 1091-1134 (2002). In some embodiments, the NO donor is a N-diazeniumdiolate (i.e., a 1-amino-substituted deazen-1-ium-1,2-diolate). N-Diazeniumdiolates are particularly attractive as NO donors due to their ability to generate NO spontaneously under biological conditions. See Hrabie, J. A. and Keefer, L. K. Chem. Rev., 102, 1135-1154 (2002); and Napoli, C. and Ianarro, L. J., Annu. Rev. Pharmacol. Toxicol., 43, 97-123 (2003). Several N-diazeniumdiolate compounds have been synthesized using a range of nucleophilic residues that encompass primary and secondary amines, polyamines, and secondary amino acids, See Hrabie, J. A., and Keefer L. K. Chem. Rev., 102, 1135-1154 (2002). In the formation of the N-diazeniumdiolate, one equivalent of amine reacts with two equivalents of nitric oxide under elevated pressure. A base (e.g., an alkoxide like methoxide) removes a proton from the amine nitrogen to create the anionic, stabilized N(O)NO group. While stable under ambient conditions, N-diazeniumdiolates decompose spontaneously in aqueous media to generate NO at rates dependent upon pH, temperature, and/or the structure of the amine moiety.

In some cases, the first component can include at least one nitric oxide releasing compound having at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

As stated above, the compounds described herein can include at least one nitric oxide releasing functional group. Although various NO donors (e.g., diazeniumdiolates, S-nitrosothiols, metal nitrosyls, organic nitrates) are known to provide for controlled exogenous NO release, the diazeniumdiolate functional group (NONOate) in the compounds disclosed herein are attractive because of their good stability and facile storage, and because they spontaneously undergo proton-triggered dissociation under physiological conditions to regenerate nitric oxide. Certain compounds include two diazeniumdiolate groups on one carbon atom, each having a charge and each with an associated pharmaceutically-acceptable cation to balance the charge on the diazeniumdiolate groups. The compounds are small molecules (having a molecular weight of 500 g/mol or less, without the cation, as further described below) that release nitric oxide (NO) and exhibit antimicrobial characteristics, including antiviral, antibacterial, and antifungal characteristics, anti-inflammatory properties, and other beneficial therapeutic properties. In some examples, the diazeniumdiolate is an N-diazeniumdiolate. In some examples, the diazeniumdiolate is a C-diazeniumdiolate

Optionally, the compound has the following structure, as represented by Formula I:

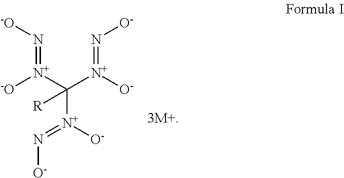

In Formula I, R is hydrogen, deuterium, C₁₋₁₂ alkyl, aryl, heteroaryl, alkylaryl, arylalkyl, or carbonyl. Optionally R is substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H, wherein a, b, c, d, e, and f are each independently selected from an integer of 0, 1, 2, 3, 4, 5, 6, 7, 8, 9, or 10.

Additionally in Formula I, M₊ is a cation. For example, M₊ can be a pharmaceutically acceptable cation. Optionally, the cation is selected from the group consisting of sodium, potassium, lithium, calcium, magnesium, and quaternary ammonium salts (e.g., ammonium or substituted ammonium).

In these compositions, a ratio of the compound to the cation is such that the overall net charge of the compound is neutral. In cases where M₊ is a cation with a valence other than one, a ratio of the compound to the cation is such that the total positive charge equals the total negative charge. By way of example, for a compound having a total charge of negative three, and a cation with a total charge of positive one, there would be one compound and three cations.

For example, the compound can be represented by Structure I-A, as shown below:

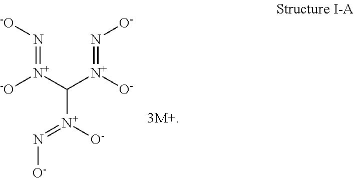

As shown above in Structure I-A, the compound has a total charge of negative three. Therefore, three cations (i.e., 3 M₊) are present to balance the charge of the compound (i.e., the total positive charge equals the total negative charge).

An example of Structure I-A includes the following compound:

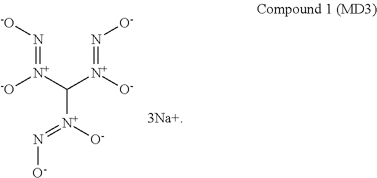

The compound can have a molecular weight below 500 g/mol, not including the associated cation (e.g., the associated pharmaceutically-acceptable cation). For example, the compound can have a molecular weight of 450 g/mol or less, 400 g/mol or less, 350 g/mol or less, 300 g/mol or less, 250 g/mol or less, or 200 g/mol or less. Optionally, the molecular weight of the compound, excluding the associated cation, can be from 100 g/mol to below 500 g/mol, from 120 g/mol to 450 g/mol, from 150 g/mol to 400 g/mol, or from 175 g/mol to 350 g/mol.

Additional details regarding the mechanism of action of the compounds described herein, including their nitric oxide delivery properties, and advantageous properties of the compounds (e.g., storage stability) are described in PCT/US2021/016841, entitled “Nitric Oxide-Releasing Antibacterial Compounds, Formulations, and Methods Pertaining Thereto;” PCT/US2021/016854, entitled “Nitric Oxide-Releasing Antibacterial Compounds, Formulations, and Methods Pertaining Thereto;” and/or PCT/US2021/016869, entitled “Nitric Oxide-Releasing Antibacterial Compounds, Formulations, and Methods Pertaining Thereto;” each of which are incorporated herein by reference in their entireties.

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alkyl, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ alkynyl.

Heteroalkyl, heteroalkenyl, and heteroalkynyl are defined similarly as alkyl, alkenyl, and alkynyl, but can contain O, S, or N heteroatoms or combinations thereof within the backbone. Ranges of these groups useful with the compounds and methods described herein include C₁-C₂₀ heteroalkyl, C₂-C₂₀ heteroalkenyl, and C₂-C₂₀ heteroalkynyl. Additional ranges of these groups useful with the compounds and methods described herein include C₁-C₁₂ heteroalkyl, C₂-C₁₂ heteroalkenyl, C₂-C₁₂ heteroalkynyl, C₁-C₆ heteroalkyl, C₂-C₆ heteroalkenyl, C₂-C₆ heteroalkynyl, C₁-C₄ heteroalkyl, C₂-C₄ heteroalkenyl, and C₂-C₄ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₀ cycloalkyl, C₃-C₂₀ cycloalkenyl, and C₃-C₂₀ cycloalkynyl. Additional ranges of these groups useful with the compounds and methods described herein include C₅-C₁₂ cycloalkyl, C₅-C₁₂ cycloalkenyl, C₅-C₁₂ cycloalkynyl, C₅-C₆ cycloalkyl, C₅-C₆ cycloalkenyl, and C₅-C₆ cycloalkynyl.

The terms heterocycloalkyl, heterocycloalkenyl, and heterocycloalkynyl are defined similarly as cycloalkyl, cycloalkenyl, and cycloalkynyl, but can contain O, S, or N heteroatoms or combinations thereof within the cyclic backbone. Ranges of these groups useful with the compounds and methods described herein include C₃-C₂₀ heterocycloalkyl, C₃-C₂₀ heterocycloalkenyl, and C₃-C₂₀ heterocycloalkynyl. Additional ranges of these groups useful with the compounds and methods described herein include C₅-C₁₂ heterocycloalkyl, C₅-C₁₂ heterocycloalkenyl, C₅-C₁₂ heterocycloalkynyl, C₅-C₆ heterocycloalkyl, C₅-C₆ heterocycloalkenyl, and C₅-C₆ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, and pyrazine. Aryl and heteroaryl molecules can also include additional fused rings, for example, benzofuran, indole, benzothiophene, naphthalene, anthracene, and quinoline. The aryl and heteroaryl molecules can be attached at any position on the ring, unless otherwise noted.

The term alkoxy as used herein is an alkyl group bonded through a single, terminal ether linkage. The term aryloxy as used herein is an aryl group bonded through a single, terminal ether linkage. Likewise, the terms alkenyloxy, alkynyloxy, heteroalkyloxy, heteroalkenyloxy, heteroalkynyloxy, heteroaryloxy, cycloalkyloxy, and heterocycloalkyloxy as used herein are an alkenyloxy, alkynyloxy, heteroalkyloxy, heteroalkenyloxy, heteroalkynyloxy, heteroaryloxy, cycloalkyloxy, and heterocycloalkyloxy group, respectively, bonded through a single, terminal ether linkage.

The term hydroxy as used herein is represented by the formula —OH.

The terms amine or amino as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The alkoxy, aryloxy, amino, alkyl, alkenyl, alkynyl, aryl, heteroalkyl, heteroalkenyl, heteroalkynyl, heteroaryl, cycloalkyl, or heterocycloalkyl molecules used herein can be substituted or unsubstituted. As used herein, the term substituted includes the addition of an alkoxy, aryloxy, amino, alkyl, alkenyl, alkynyl, aryl, heteroalkyl, heteroalkenyl, heteroalkynyl, heteroaryl, cycloalkyl, or heterocycloalkyl group to a position attached to the main chain of the alkoxy, aryloxy, amino, alkyl, alkenyl, alkynyl, aryl, heteroalkyl, heteroalkenyl, heteroalkynyl, heteroaryl, cycloalkyl, or heterocycloalkyl, e.g., the replacement of a hydrogen by one of these molecules. Examples of substitution groups include, but are not limited to, hydroxy, halogen (e.g., F, Br, Cl, or I), and carboxyl groups. Conversely, as used herein, the term unsubstituted indicates the alkoxy, aryloxy, amino, alkyl, alkenyl, alkynyl, aryl, heteroalkyl, heteroalkenyl, heteroalkynyl, heteroaryl, cycloalkyl, or heterocycloalkyl has a full complement of hydrogens, i.e., commensurate with its saturation level, with no substitutions, e.g., linear decane (—(CH₂)₉—CH₃).

The C-diazeniumdiolates described herein are pH-triggered NO-releasing donors (also referred to herein as NO-releasing compounds or NO-releasing agents). Reacting with protons under physiological conditions (e.g., 37° C., pH 7.4), 1 mole of Compound 1 (MD3) generates two moles of NO and 2 to 3 moles of nitroxyl compounds.

In several embodiments, the NO-releasing compounds are stable at a variety of temperatures from frozen to room temperature 25° C. (e.g., −20° C., 0° C., 5° C., 20° C., etc.) and are stable for prolonged storage periods (e.g., 10 hours, 20 hours, 22 hours, 25 hours, 30 hours, etc., days such as 1 day, 3 days, 5 days, 6 days, 7 days, 15 days, 30 days, 45 days, etc., weeks such as 1 week, 2 weeks, 3 weeks, 4 weeks, 6 weeks, 8 weeks, etc., months such as 1 month, 2 months, 3 months, 4 months, 5 months, 6 months, etc., or even years (1 year, 2 years, or greater)).

In some cases, the compound has a total releasable NO storage in a range of 0.1 μmol to 23.0 μmol of NO per mg of the compound (e.g., from 0.1 μmol to 15 μmol per mg of the compound, from 0.5 μmol to 7.5 μmol per mg of the compound, from 1 μmol to 7.0 μmol per mg of the compound, from 1.5 μmol to 6.5 μmol per mg of the compound, from 2.0 μmol to 6.0 μmol per mg of the compound, from 2.5 μmol to 5.5 μmol per mg of the compound, or from 3.0 μmol to 5.0 μmol per mg of the compound). For example, the total releasable NO storage of the compounds for use in the composition can be 0.1 μmol, 0.2 μmol, 0.3 μmol, 0.4 μmol, 0.5 μmol, 0.6 μmol, 0.7 μmol, 0.8 μmol, 0.9 μmol, 1.0 μmol, 1.1 μmol, 1.2 μmol, 1.3 μmol, 1.4 μmol, 1.5 μmol, 1.6 μmol, 1.7 μmol, 1.8 μmol, 1.9 μmol, 2.0 μmol, 2.1 μmol, 2.2 μmol, 2.3 μmol, 2.4 μmol, 2.5 μmol, 2.6 μmol, 2.7 μmol, 2.8 μmol, 2.9 μmol, 3.0 μmol, 3.1 μmol, 3.2 μmol, 3.3 μmol, 3.4 μmol, 3.5 μmol, 3.6 μmol, 3.7 μmol, 3.8 μmol, 3.9 μmol, 4.0 μmol, 4.1 μmol, 4.2 μmol, 4.3 μmol, 4.4 μmol, 4.5 μmol, 4.6 μmol, 4.7 μmol, 4.8 μmol, 4.9 μmol, 5.0 μmol, 5.1 μmol, 5.2 μmol, 5.3 μmol, 5.4 μmol, 5.5 μmol, 5.6 μmol, 5.7 μmol, 5.8 μmol, 5.9 μmol, 6.0 μmol, 6.1 μmol, 6.2 μmol, 6.3 μmol, 6.4 μmol, 6.5 μmol, 6.6 μmol, 6.7 μmol, 6.8 μmol, 6.9 μmol, 7.0 μmol, 7.1 μmol, 7.2 μmol, 7.3 μmol, 7.4 μmol, 7.5 μmol, 7.6 μmol, 7.7 μmol, 7.8 μmol, 7.9 μmol, 8.0 μmol, 8.1 μmol, 8.2 μmol, 8.3 μmol, 8.4 μmol, 8.5 μmol, 8.6 μmol, 8.7 μmol, 8.8 μmol, 8.9 μmol, 9.0 μmol, 9.1 μmol, 9.2 μmol, 9.3 μmol, 9.4 μmol, 9.5 μmol, 9.6 μmol, 9.7 μmol, 9.8 μmol, 9.9 μmol, 10.0 μmol, 10.1 μmol, 10.2 μmol, 10.3 μmol, 10.4 μmol, 10.5 μmol, 10.6 μmol, 10.7 μmol, 10.8 μmol, 10.9 μmol, 11.0 μmol, 11.1 μmol, 11.2 μmol, 11.3 μmol, 11.4 μmol, 11.5 μmol, 11.6 μmol, 11.7 μmol, 11.8 μmol, 11.9 μmol, 12.0 μmol, 12.1 μmol, 12.2 μmol, 12.3 μmol, 12.4 μmol, 12.5 μmol, 12.6 μmol, 12.7 μmol, 12.8 μmol, 12.9 μmol, 13.0 μmol, 13.1 μmol, 13.2 μmol, 13.3 μmol, 13.4 μmol, 13.5 μmol, 13.6 μmol, 13.7 μmol, 13.8 μmol, 13.9 μmol, 14.0 μmol, 14.1 μmol, 14.2 μmol, 14.3 μmol, 14.4 μmol, 14.5 μmol, 14.6 μmol, 14.7 μmol, 14.8 μmol, 14.9 μmol, 15.0 μmol, 15.1 μmol, 15.2 μmol, 15.3 μmol, 15.4 μmol, 15.5 μmol, 15.6 μmol, 15.7 μmol, 15.8 μmol, 15.9 μmol, 16.0 μmol, 16.1 μmol, 16.2 μmol, 16.3 μmol, 16.4 μmol, 16.5 μmol, 16.6 μmol, 16.7 μmol, 16.8 μmol, 16.9 μmol, 17.0 μmol, 17.1 μmol, 17.2 μmol, 17.3 μmol, 17.4 μmol, 17.5 μmol, 17.6 μmol, 17.7 μmol, 17.8 μmol, 17.9 μmol, 18.0 μmol, 18.1 μmol, 18.2 μmol, 18.3 μmol, 18.4 μmol, 18.5 μmol, 18.6 μmol, 18.7 μmol, 18.8 μmol, 18.9 μmol, 19.0 μmol, 19.1 μmol, 19.2 μmol, 19.3 μmol, 19.4 μmol, 19.5 μmol, 19.6 μmol, 19.7 μmol, 19.8 μmol, 19.9 μmol, 20.0 μmol, 20.1 μmol, 20.2 μmol, 20.3 μmol, 20.4 μmol, 20.5 μmol, 20.6 μmol, 20.7 μmol, 20.8 μmol, 20.9 μmol, 21.0 μmol, 21.1 μmol, 21.2 μmol, 21.3 μmol, 21.4 μmol, 21.5 μmol, 21.6 μmol, 21.7 μmol, 21.8 μmol, 21.9 μmol, 22.0 μmol, 22.1 μmol, 22.2 μmol, 22.3 μmol, 22.4 μmol, 22.5 μmol, 22.6 μmol, 22.7 μmol, 22.8 μmol, 22.9 μmol, or 23.0 μmol per mg of the compound.

The compound can have a total duration of NO release, upon activation, in a range of 0.1 to 60 hours. In some cases, the NO release may occur over a period of about 0.1 hours, 0.25 hours, 0.5 hours, 1 hour, 2 hours, 3 hours, 4 hours, 5 hours, 10 hours, 15 hours, 20 hours, 24 hours, 36 hours, 48 hours, or 60 hours. In some embodiments, within 2 hours of being added to a phosphate buffered saline (PBS) buffer solution, the compounds release greater than or equal to about: 25%, 50%, 75%, 85%, 90%, 95%, 100%, or ranges including and/or spanning the aforementioned values, their total wt. % of bound NO. Optionally, the compound has a total NO release of 0.1-8.0 μmol of NO per mg of the compound after 4 hours of the initiation of NO release (also referred to as “activation”).

In some embodiments, the compounds have a release rate per hour using chemiluminescent based nitric oxide detection of less than or equal to about: 0.2%, 0.5%, 1.0%, 1.5%, 2.5%, 5.0%, 10%, or ranges including and/or spanning the aforementioned values.

Optionally, the compound for use in the compositions described herein has a NO release half-life in the range of 0.01-24 hours. In several embodiments, the NO release half-life is equal to or at least about: 0.01 hours, 0.1 hours, 0.25 hours, 0.5 hours, 1 hour, 2 hours, 3 hours, 4 hours, 5 hours or ranges including and/or spanning the aforementioned values. In some embodiments, the NO release occurs in less than or equal to about: 0.01 hours, 0.1 hours, 0.25 hours, 0.5 hours, 1 hour, 2 hours, 3 hours, 4 hours, 5 hours, 10 hours, 15 hours, 20 hours, 24 hours or ranges including and/or spanning the aforementioned values.

b. Additional Active Agent

As described above, the compositions described herein can include an additional active agent. Optionally, the additional active agent can include one or more of a peptide, a protein, a corticosteroid, an antibiotic, a short acting beta agonist, a long acting beta agonist, an anti-cholinergic, an antiviral, a monoclonal antibody (mAb), and an antifungal.

The additional active agent can be, in some instances, an antibiotic. Suitable antibiotics can include any antibiotic effective for treating a bacterial infection and/or inhibiting or disrupting a biofilm and include, for example, tetracyclines (e.g., minocycline), quinolones (e.g., ciprofloxacin, levofloxacin, and nalidixic acid), aminoglycosides (e.g., amikacin, gentamycin, kanamycin, and tobramycin), carbapenems (e.g., meropenem), cephalosporins (e.g., ceftriaxone and ceftazidime), macrolides (e.g., erythromycin and clarithromycin), polypeptides (e.g., colistin and polymxin B), sulfonamides (e.g., sulfamethoxazole), glycylcyclines (e.g., tigecycline), beta lactams (e.g., penams), lipopeptides (e.g., daptomycin), oxazolidinones (e.g., linezolid), trimethoprim, and monobactams.

For example, the additional active agent described herein can include an antibiotic, such as acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; aztreonam; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; Lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodium; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin; oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillin G benzathine; penicillin G potassium; penicillin G procaine; penicillin G sodium; penicillin V; penicillin V benzathine; penicillin V hydrabamine; penicillin V potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin B sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; or zorbamycin.

In some cases, the additional active agent can be an antifungal agent. Optionally, the antifungal agent can be selected from the group consisting of a polyene, an azole, an allylamine, and an echinocandin. Exemplary antifungal agents include, for example, amphoterican B, nystatin, flucytosin, natamycin, ketoconazole, econoazole, miconazole, itraconazole, fluconazole, clotrimazole, griseofulvin, oxiconazole, terconazole, tioconazole, clotrimazole, silver sulfadiazine, ciclopirox olamine, and terbinafine.

Optionally, the additional active agent is a short acting beta agonist. In some cases, the short acting beta agonist can be selected from albuterol, epinephrine, pirbuterol, levalbuterol, metaproteronol, and maxair.

In some cases, the additional active agent is a long-acting beta agonist, such as salmeterol, formoterol, arformoterol, clenbuterol, tulobuterol, vilanterol, indacaterol, carmoterol, isoproterenol, procaterol, bambuterol, milveterol, or olodaterol.

Optionally, the additional active agent is a corticosteroid. In some cases, the corticosteroid can be budesonide, fluticasone, flunisolide, triamcinolone, beclomethasone, mometasone, ciclesonide, or dexamethasone.

The additional active agent can optionally be an anti-cholinergic agent, such as ipratropium bromide or oxitropium bromide.

In some cases, the additional active agent can be an antiviral, such as pegylated interferon-α, ribavirin, nucleoside polymerase inhibitors, non-nucleoside polymerase inhibitors, protease inhibitors, nucleoside or nucleotide reverse transcriptase inhibitors, non-nucleoside reverse transcriptase inhibitors, entry inhibitors, assembly inhibitors, integrase inhibitors, kinase inhibitors, enzyme inhibitors, maturation inhibitors, M2 inhibitors, and neuraminidase inhibitors. Examples of such additional antivirals include, but are not limited to abacavir, acyclovir, acyclovir, adefovir, amantadine, amprenavir, ampligen, arbidol, atazanavir, atripla, boceprevir, cidofovir, combivir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, nelfinavir, nevirapine, nexavir, oseltamivir, peginterferon alfa-2a, penciclovir, peramivir, pleconaril, podophyllotoxin, raltegravir, ribavirin, rimantadine, ritonavir, pyramidine, saquinavir, stavudine, tenofovir, tenofovir disoproxil, tipranavir, trifluridine, trizivir, tromantadine, truvada, valaciclovir, valganciclovir, vicriviroc, vidarabine, viramidine zalcitabine, zanamivir, and/or zidovudine.

In some cases, the additional active agent can be peptide, such as hormonal peptides (e.g., insulin, oxytocin, semaglutide), anticancer peptides, antimicrobial peptides, cell-penetrating peptides, exenatide, liraglutide, lixisenatide, albiglutide, dulaglutide, linaclotide, lanreotide, salmon calcitonin, bivalirudin, endothelin, melittin, glucagon, or any combination thereof.

In some cases, the additional active agent can be a protein, such as monoclonal antibodies, peptide hormones, growth factors, plasma proteins, enzymes, hemolytic factors, recombinant proteins, or any combination thereof.

In some cases, the additional active agent can be a monoclonal antibody (mAb), such as adalimumab, rituximab, trastuzumab, nivolumab, pembrolizumab, infliximab, ocrelizumab, basiliximab, omalizumab, palivizumab, daclizumab, belimumab, tocilizumab, ustekinumab, bevacizumab, cetuximab, brentuximab vedotin, or any combination thereof.

c. Optional Additives

The compositions described herein can further include one or more additives. The one or more additives can include, for example, one or more carbohydrates, organic salts, amino acids, preservatives, salts, chelators, viscosity modifiers, stabilizers, surfactants, antioxidants (e.g., N-acetylcysteine or glutathione), polymers, mucolytics, glycols, and/or cosolvents. The compositions can also contain wetting or emulsifying agents, lubricants, glidants, emollients, humectants, thickeners, and/or flavoring agents. In some cases, the optional additives exclude buffering agents.

In some cases, the one or more additives can include viscosity modifiers (e.g., viscosity-reducing agents), natural and synthetic anti-biofilm agents (e.g., chitosan), biofilm dispersing agents, natural and synthetic anti-quorum-sensing agents (e.g., autoinducer-2 or N-acyl homo-serine lactones), siderophores, iron chelators, iron mimetics (e.g., gallium (Ga) and gallium-containing compounds, such as gallium azoles (Ga-azoles)), anti-persister cell agents (e.g., 4-(4,7-di-methyl-1,2,3,4-tetrahydro-naphthalene-1-yl) pentanoic acid (DMNP)), antimicrobial peptides (AMPs) (e.g., LL-37 or lactoferricin), efflux pump inhibitors, and/or bacteriophage therapy.

In some cases, the one or more additives can include carbohydrates, such as glucose, lactose, cellulose, polysaccharides, simple monosaccharides, or any combination thereof.

In some cases, the one or more additives can include organic salts, such as acetates, citrates, lactates, oxalates, tartrates, stearates, formats, benzoates, maleates, malates, gluconates, malonates, sorbates, glycerates, urates, and/or salicylates. Exemplary organic salts may include but are not limited to sodium acetate, sodium citrate, sodium lactate, calcium oxalate, potassium tartrate, sodium stearate, sodium formate, sodium benzoate, sodium malate, sodium gluconate, sodium malonate, sodium sorbate, sodium glycerate, sodium urate, sodium salicylate, or any combination thereof.

In some cases, the one or more additives can include amino acids, such as alanine, arginine, asparagine, aspartic acid, cysteine, glutamic acid, glutamine, glycine, histidine, isoleucine, leucine, lysine, methionine, phenylalanine, threonine, tryptophan, valine, derivatives thereof, other similar amino acids, or any combination thereof.

In some cases, the one or more additives can include preservatives. Any pharmaceutically acceptable preservative known to those of ordinary skill in the art is contemplated for inclusion in the present compositions. Examples of such preservatives include methylparaben, methylparaben sodium, propylparaben, propylparaben sodium, benzalkonium chloride, benzethonium chloride, or any combination thereof.

In some cases, the one or more additives can include chelators, such as deferasirox, deferiprone, deferoxamine, dimercaprol, penicillamine, 2,3-dimercapto-1-propanesulfonic acid, ethylenediaminetetraacetic acid, 1,10-phenanthroline, citric acid, dimercaptosuccinic acid, sodium calcium edetate, pentetic acid, picolinic acid, or any combination thereof.

In some cases, the one or more additives can include viscosity modifiers, such as natural gums (e.g., acacia gun, xanthan gum), cellulose derivatives (e.g., methylcellulose, carboxymethylcellulose (CMC), hydroxypropyl methylcellulose (HMPC)), synthetic polymers (e.g., polyvinylpyrrolidone (PVP), polyethylene glycol (PEG)), microcrystalline cellulose, chitosan, clays, acids, or any combination thereof.

In some cases, the one or more additives can include stabilizers, such as polymers (e.g., polyvinyl alcohol (PVA), hydroxypropyl cellulose (HPC), hydroxyethyl cellulose (HEC), carboxymethylcellulose sodium (NaCMC)), surfactants (e.g., Tween 20, Poloxamer 188, docusate sodium (SD), sodium lauryl sulfate (SLS)), sugar alcohols (e.g., sorbitol, mannitol), sugars (e.g., sucrose), buffers, vitamin E, or any combination thereof.

In some cases, the one or more additives can include mucolytics, such as thiol-containing mucolytics (e.g., acetylcysteine), thioester mucolytics (e.g., carbocisteine, erdosteine, fudosteine), peptide mucolytics (e.g., thymosin beta-4), DNAse mucolytics, bromohexine, ambroxol, N-acetylcysteine (NAC), sobrerol, or any combination thereof.

In some cases, the one or more additives can include glycols, such as polyethylene glycol (PEG), propylene glycol, diethylene glycol, ethylene glycol, hexylene glycol, or any combination thereof.

In some cases, the one or more additives can include co-solvents, such as ethanol, propylene glycol, polyethylene glycol, glycerin (glycerol), methylpyrrolidone, dimethyl sulfoxide (DMSO), or any combination thereof.

Optionally, the additives can be present in the compositions in an amount of less than 1 wt. %. For example, the amount of the additive can be less than 0.9 wt. %, less than 0.8 wt. %, less than 0.7 wt. %, less than 0.6 wt. %, less than 0.5 wt. %, less than 0.4 wt. %, less than 0.3 wt. %, less than 0.2 wt. %, or less than 0.1 wt. %. The amount of additive can optionally be 0.1 to 0.9 wt. %, 0.2 to 0.8 wt. %, or 0.3 to 0.7 wt. %.

Viscosity modifiers can optionally be included in the compositions as described herein. Optionally, the viscosity modifiers can be included in the compositions in an amount of up to 5 wt. % (e.g., 0.1 wt. % to 5 wt. %, 0.5 wt. % to 4.5 wt. %, 1.0 wt. % to 4.0 wt. %, 1.5 wt. % to 3.5 wt. %, or 2.0 wt. % to 3.0 wt. %). For example, the viscosity modifier can be 0.1 wt. %, 0.5 wt. %, 1.0 wt. %, 1.5 wt. %, 2.0 wt. %, 2.5 wt. %, 3.0 wt. %, 3.5 wt. %, 4.0 wt. %, or 4.5 wt. %, or 5.0 wt. %.

### II. Methods of Making the Compounds

The compounds described herein can be prepared in a variety of ways. The compounds can be synthesized using, for example, various synthetic methods. At least some of these methods are known in the art of synthetic organic chemistry. The compounds described herein can be prepared from readily available starting materials. Optimum reaction conditions may vary with the particular reactants or solvents used, but such conditions can be determined by one skilled in the art.

Variations on Formula I and the compounds described herein include the addition, subtraction, or movement of the various constituents as described for each compound. Similarly, when one or more chiral centers are present in a molecule, all possible chiral variants are included. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts, Greene's Protective Groups in Organic Synthesis, 5th. Ed., Wiley & Sons, 2014, which is incorporated herein by reference in its entirety.

Reactions to produce the compounds described herein can be carried out in solvents, which can be selected by one of ordinary skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H-NMR or ¹³C-NMR), infrared spectroscopy (IR), spectrophotometry (e.g., UV-visible), or mass spectrometry (MS), or by chromatography such as high performance liquid chromatography (HPLC) or thin layer chromatography (TLC).

Optionally, the compounds described herein can be prepared according to the methods of synthesis described in PCT/US2021/016841, entitled “Nitric Oxide-Releasing Antibacterial Compounds, Formulations, and Methods Pertaining Thereto;” PCT/US2021/016854, entitled “Nitric Oxide-Releasing Antibacterial Compounds, Formulations, and Methods Pertaining Thereto;” and/or PCT/US2021/016869, entitled “Nitric Oxide-Releasing Antibacterial Compounds, Formulations, and Methods Pertaining Thereto;” each of which are incorporated herein by reference in their entireties.

### III. Pharmaceutical Compositions

In some cases, the composition is a pharmaceutical composition. In cases where the composition is a pharmaceutical composition, the compositions can include one or more of the compounds described herein and a pharmaceutically acceptable carrier. Furthermore, the one or more compounds described herein can be combined with other agents, including treatments for lung, digestive, hepatic, and biliary tract related diseases and disorders. For example, in the case of cystic fibrosis, the compositions described herein can be combined with mucus thinning drugs (e.g., dornase alfa, N-acetyl cysteine, and hypertonic saline), bronchodilators (e.g., metaproterenol sulfate, pirbuterol acetate, salmeterol, albuterol, and terbutaline sulfate), P2Y2-receptor agonists (e.g., denufosol), and agents that target nonsense mutations (e.g., PTC124). Further examples of additional agents that can be combined with the compounds described herein include antibiotics (e.g., aminoglycosides, antipseudomonal penicillins, and cephalosporins), antimicrobial drugs (e.g., rifabutin), ethambutol, clarithromycin, clofazimine, aztreonam, steroidal and nonsteroidal anti-inflammatory drugs (e.g., ibuprofen and prednisone), pentoxifylline, dornase alfa, or ursodeoxycholic acid.

The one or more compounds described herein, with or without additional agents, can be provided in the form of an inhaler or nebulizer for inhalation therapy. In some cases, the one or more compounds described herein, with or without additional agents, can be administered using a metered dose inhaler or a dry powder inhaler. As used herein, inhalation therapy refers to the delivery of a therapeutic agent, such as the compounds described herein, in an aerosol form to the respiratory tract (i.e., pulmonary delivery). As used herein, the term aerosol refers to very fine liquid or solid particles suspended in a gas (e.g., air) and delivered to a site of therapeutic application. When a pharmaceutical aerosol is employed, the aerosol contains the one or more compounds described herein, which can be dissolved, suspended, or emulsified in a mixture of a fluid carrier and/or a propellant, in the case of a metered dose inhaler. The aerosol can be in the form of a solution, suspension, emulsion, powder, or semi-solid preparation. Aerosols employed are intended for administration as fine, solid particles or as liquid mists via the respiratory tract of a patient.

As described above, the one or more compositions described herein can be provided with a nebulizer, which is an instrument that generates very fine liquid particles of substantially uniform size in a gas. The liquid containing the one or more compounds described herein can be dispersed as droplets about 5 mm or less in geometric diameter (e.g., 5 μm or less or 1 μm to 5 μm in geometric diameter) in the form of a mist. The small droplets can be carried by a current of air or oxygen through an outlet tube of the nebulizer. The resulting mist can penetrate into the respiratory tract of the patient.

Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

The preparation of pharmaceutically acceptable carriers and formulations containing these materials is described in, e.g., Remington: The Science and Practice of Pharmacy, Adeboye Adejare ed., 23rd Ed., Academic Press (2021). Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, New Jersey), polyethylene glycol (PEG), and PLURONICS™ (BASF; Florham Park, NJ).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the compounds described herein or derivatives thereof include ointments, powders, sprays, inhalants, gels, pastes, creams, and lotions. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

As noted above, the compositions can include one or more of the compounds described herein or pharmaceutically acceptable salts thereof. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein.

The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein.)

Administration of the compounds and compositions described herein or pharmaceutically acceptable salts thereof can be carried out using therapeutically effective amounts of the compounds and compositions described herein or pharmaceutically acceptable salts thereof as described herein for periods of time effective to treat a disorder. The effective amount of the compounds and compositions described herein or pharmaceutically acceptable salts thereof as described herein may be determined by one of ordinary skill in the art and includes exemplary dosage amounts for a mammal of from about 0.01 to about 200 mg/kg of body weight of active compound per day, which may be administered in a single dose or in the form of individual divided doses, such as from 1 to 4 times per day. Alternatively, the dosage amount can be from about 0.05 to about 190 mg/kg of body weight of active compound per day, about 0.1 to about 180 mg/kg of body weight of active compound per day, about 0.25 to about 175 mg/kg of body weight of active compound per day, about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

### IV. Inhalation Formulations

The compositions and pharmaceutical compositions described herein are used to prepare formulations suitable for administration by inhalation. The formulations described herein can be prepared from aqueous based compositions and dry powder compositions.

a. Aqueous Based Compositions

A formulation as described herein can be prepared from an aqueous based composition including a nitric oxide (NO) releasing compound and an aqueous carrier. The composition can further include a pharmaceutically acceptable carrier, along with one or more additives and/or one or more additional active agents as described herein. The formulations can be used to treat a respiratory disease in a subject, by administering an effective amount of the formulation to the subject. Optionally, the aqueous compositions can be contained in a drug administration device, such as a container attached to a pump, a spray, or a soft mist inhaler. As understood by a person of ordinary skill in the art, such aqueous compositions, when delivered by the indicated devices, are aerosolized into a gas stream.

In some cases, an aqueous based composition includes a nitric oxide (NO) releasing compound and a propellant. The propellant can be a fluorinated hydrocarbon, such as 1,1,1,2-tetrafluoroethane, 1,1,1,2,3,3,3-heptafluoropropane, trans-1,3,3,3-tetrafluoropropene, 1,1-difluoroethane, or a combination thereof, or any other suitable propellant as known to those of ordinary skill in the art. The composition can also include ethanol.

The composition can further include a pharmaceutically acceptable carrier, along with one or more additives and/or one or more additional active agents as described herein. The formulations can be used to treat a respiratory disease in a subject, by administering an effective amount of the formulation to the subject. Optionally, the aqueous compositions can be contained in a drug administration device, such as an inhaler (e.g., a metered dose inhaler) or other propellant administration device. As understood by a person of ordinary skill in the art, such aqueous compositions, when delivered by the indicated devices, are aerosolized into a gas stream.

In other cases, an aqueous based composition includes a nitric oxide (NO) releasing compound and an aqueous based vaporizable liquid. The aqueous based vaporizable liquid can include water. The aqueous based vaporizable liquid can also include one or more of propylene glycol and vegetable glycerine, or any other suitable aqueous based vaporizable liquid as known to those of ordinary skill in the art.

The composition can further include one or more additives and/or one or more additional active agents as described herein. The formulations can be used to treat a respiratory disease in a subject, by administering an effective amount of the formulation to the subject. Optionally, the aqueous compositions can be contained in a drug administration device, such as an electronic vaporizing device. As understood by a person of ordinary skill in the art, such aqueous compositions, when delivered by the indicated devices, are vaporized for delivery.

b. Dry Powder Formulations

A formulation as described herein can be prepared from a powder based composition including a nitric oxide (NO) releasing compound and a carrier particle. The carrier particle can be, for example, lactose or a stearate treated lactose. The stearate treated lactose can optionally include a metal cationic salt, such as a magnesium salt, a potassium salt, a sodium salt, or a calcium salt. The composition can further include one or more additives and/or one or more additional active agents as described herein.

The formulations can be used to treat a respiratory disease in a subject, by administering an effective amount of the formulation to the subject. Optionally, the aqueous compositions can be contained in a drug administration device, such as a dry powder inhaler. As understood by a person of ordinary skill in the art, such compositions, when delivered by the indicated devices, are aerosolized.

In some cases, a powder based composition includes a porous particle comprising a nitric oxide (NO) releasing compound. In some cases, the porous particle has a mass median aerodynamic diameter of no greater than 10 microns (e.g., no greater than 8 microns in diameter, no greater than 5 microns in diameter, no greater than 3 microns in diameter, or no greater than 2 microns in diameter, all referring to mass median aerodynamic diameter). In some cases, the porous particle is spherical in shape (also referred to as a porous sphere). The composition can further include one or more additives and/or one or more additional active agents as described herein.

The formulations can be used to treat a respiratory disease in a subject, by administering an effective amount of the formulation to the subject. Optionally, the aqueous compositions can be contained in a drug administration device, such as a dry powder inhaler. As understood by a person of ordinary skill in the art, such compositions, when delivered by the indicated devices, are aerosolized.

### V. Methods of Use

Provided herein are methods of administering the compositions described herein to a subject. As used herein, the terms “administering” and “administration” refer to any method of providing composition to a subject. Such methods are well known to those skilled in the art and include, but are not limited to, administration by inhalation, oral administration, transdermal administration, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. In some examples described herein, the administration is performed by inhalation.

Administration can be continuous or intermittent. The compositions described herein can be administered therapeutically; that is, administered to treat an existing disease or condition. In some examples, the composition can be administered prophylactically; that is, administered for prevention of a disease or condition.

In some examples, provided herein are methods of treating a respiratory disease in a subject. The methods include administering to the subject an effective amount of a composition as described herein. The respiratory disease can include an acute or chronic lung infection caused by a microbial pathogen from one or more of bacteria, viruses, or fungi. Effective amount, when used to describe an amount of compound in a method, refers to the amount of a compound that achieves the desired pharmacological effect or other biological effect. The effective amount of the composition can be, for example, delivery of enough compound to the target region of the lung to achieve the minimal inhibitory concentration (MIC) or the minimal bactericidal concentration (MBC) against a particular bacterial strain in the epithelial lining fluid (ELF).

In some examples, the respiratory diseases are caused by bacteria in humans, e.g., pediatric and geriatric populations, and in animals, e.g., veterinary applications. In some cases, the bacteria include at least one of Gram-positive bacteria, Gram-negative bacteria, and atypical bacteria.

Optionally, the bacteria is a Gram-positive bacteria species, such as an Actinomyces species, a Bacillus species, a Clostridium species, a Corynebacterium species, an Enterococcus species, a Leuconostoc species, a Micrococcus species, a Nocardia species, a Propionibacterium species, a Staphylococcus species, or a Streptococcus species.

Optionally, the bacteria is a Gram-negative bacteria species, such as an Acinetobacter species, an Aeromonas species, an Alcaligenes/Achromobacter species, a Bacteroides species, a Bartonella species, a Bordetella species, a Borrelia species, a Brevundimonas species, a Brucella species, a Burkholderia species, a Campylobacter species, a Citrobacter species, a Coxiella species, an Ehrlichia species, an Enterobacter species, an Escherichia species, a Francisella species, a Haemophilus species, a Helicobacter species, a Klebsiella species, a Leclercia species, a Legionella species, a Leptospira species, a Listeria species, a Moraxella species, a Morganella species, a Neisseria species, an Orientia species, a Pantoea species, a Paracoccus species, a Prevotella species, a Proteus species, a Providencia species, a Pseudomonas species (e.g., Pseudomonas aeruginosa), a Ralstonia species, a Rickettsia species, a Roseomonas species, Salmonella species, a Serratia species, a Shigella species, a Sphingomonas species, a Stenotrophomonas species, a Treponema species, a Ureaplasma species, a Vibrio species, or a Yersinia species.

Optionally, the bacteria is an atypical bacteria species, such as a Mycobacteria species, a Chlamydia/Chlamidophila species, or a Mycoplasma species. In some cases, the bacteria can include antibiotic-resistant bacteria, such as antibiotic-resistant Burkholderia cepacia, carbapenem-resistant Enterobacteriaceae (CRE) gut bacteria, drug-resistant Campylobacter, drug-resistant non-typhoidal Salmonella, drug-resistant Shigella, multi-drug-resistant Acinetobacter, multi-drug-resistant Escherichia coli, multi-drug-resistant Klebsiella pneumoniae, multi-drug-resistant Neisseria gonorrhoeae, multidrug-resistant Pseudomonas aeruginosa, antibiotic-resistant Clostridium difficile, drug-resistant Streptococcus pneumoniae, clindamycin-resistant Group B Streptococcus, erythromycin-resistant Group A Streptococcus, methicillin-resistant Staphylococcus aureus (MRSA), vancomycin-resistant Staphylococcus aureus (VRSA), and vancomycin-resistant Enterococcus (VRE).

In some examples, the respiratory diseases are caused by viruses in humans, e.g., pediatric and geriatric populations, and in animals, e.g., veterinary applications. The virus can be an RNA or a DNA virus. Optionally, the virus can be an enveloped or non-enveloped virus. Examples of enveloped viruses include, but are not limited to, human immunodeficiency virus (HIV), vesicular stomatitis virus (VSV), herpes simplex viruses (HSV-1 and HSV-2), and other herpes viruses, for example, varicella-zoster virus (VZV), EBV, equine herpes virus (EHV), influenza virus and human cytomegalovirus (HCMV). Examples of non-enveloped viruses include, but are not limited to, papilloma virus (PV) and adenoviruses (AV).

In some examples, the respiratory disease is caused by a respiratory viruses including those that cause upper respiratory tract infections and lower respiratory tract infections. The viruses can include, for example, coronaviruses, rhinoviruses, Respiratory Syncytial Virus, paramyxoviruses, such as parainfluenza viruses, for example HPIV-1, HPIV-2, HPIV-3, HPIV-4, HPIV-4a and HPIV-4b, and other influenza viruses, such as influenza A and influenza B. Diseases resulting from infections by these viruses, such as common colds, influenza, bronchiolitis, pneumonia, croup, bronchitis, pharyngitis, laryngitis, otitis media and sudden acute respiratory system (SARS), can also be treated according to some of the present methods.

Optionally, the respiratory diseases are caused by fungi in humans, e.g., pediatric and geriatric populations, and in animals, e.g., veterinary applications. Representative fungal infections that can be treated include Candida albicans, drug resistant Candida albicans, Candida glabrata, Candida krusei, Candida guilliermondii, Candida auris, Candida tropicalis, Aspergillus niger, Aspergillus terreus, Aspergillus fumigatus, and/or Aspergillus flavus.

Further examples of microbial pathogens, including bacteria, viruses, or fungi, that can be the source of a respiratory disease for treatment according to the methods described herein are described in PCT/US2021/016841, entitled “Nitric Oxide-Releasing Antibacterial Compounds, Formulations, and Methods Pertaining Thereto;” PCT/US2021/016854, entitled “Nitric Oxide-Releasing Antibacterial Compounds, Formulations, and Methods Pertaining Thereto;” and/or PCT/US2021/016869, entitled “Nitric Oxide-Releasing Antibacterial Compounds, Formulations, and Methods Pertaining Thereto;” each of which are incorporated herein by reference in their entireties.

The methods of treating a respiratory disease can further include selecting a subject having a respiratory disease or at risk of developing a respiratory disease. Optionally, the subject has asthma, chronic obstructive pulmonary disease, emphysema, acute bronchitis, cystic fibrosis, pneumonia, bronchiectasis, or bronchiolitis. The methods of treatment described herein can further include treatment with one or more additional agents (e.g., an antibiotic, an antiviral, and/or an antifungal). The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

Suitable antibiotics can include any antibiotic effective for treating a respiratory disease including, for example, tetracyclines (e.g., minocycline), quinolones (e.g., ciprofloxacin, levofloxacin, and nalidixic acid), aminoglycosides (e.g., amikacin, gentamycin, kanamycin, and tobramycin), carbapenems (e.g., meropenem), cephalosporins (e.g., ceftriaxone and ceftazidime), macrolides (e.g., erythromycin and clarithromycin), polypeptides (e.g., colistin and polymxin B), sulfonamides (e.g., sulfamethoxazole), glycylcyclines (e.g., tigecycline), beta lactams (e.g., penams), lipopeptides (e.g., daptomycin), oxazolidinones (e.g., linezolid), and trimethoprim.

For example, the compounds or compositions or pharmaceutically acceptable salts thereof as described herein can be combined into a pharmaceutical composition with an additional antibiotic, such as acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; aztreonam; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; Lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodium; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin; oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillin G benzathine; penicillin G potassium; penicillin G procaine; penicillin G sodium; penicillin V; penicillin V benzathine; penicillin V hydrabamine; penicillin V potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin B sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; or zorbamycin.

Suitable antivirals include, for example, abacavir, acyclovir, acyclovir, adefovir, amantadine, amprenavir, ampligen, arbidol, atazanavir, atripla, boceprevir, cidofovir, combivir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, nelfinavir, nevirapine, nexavir, oseltamivir, peginterferon alfa-2a, penciclovir, peramivir, pleconaril, podophyllotoxin, raltegravir, ribavirin, rimantadine, ritonavir, pyramidine, saquinavir, stavudine, tenofovir, tenofovir disoproxil, tipranavir, trifluridine, trizivir, tromantadine, truvada, valaciclovir, valganciclovir, vicriviroc, vidarabine, viramidine zalcitabine, zanamivir, and/or zidovudine.

Suitable antifungals agents include, for example, amphotericin B, fluconazole, flucytosine, itraconazole, ketoconazole, clotrimazole, econozole, griseofulvin, miconazole, nystatin, and/or ciclopirox.

Optionally, the respiratory disease can be caused by a respiratory infectious viruses (e.g., infectious diseases due to respiratory infectious viruses such as influenza virus, rhino virus, corona virus, parainfluenza virus, RS virus, adeno virus, reovirus and the like), herpes zoster caused by herpes virus, diarrhea caused by rotavirus, viral hepatitis, AIDS and the like. The bacterial infectious disease is not particularly limited and includes, for example, infectious diseases caused by Bacillus cereus, Vibrio parahaemolyticus, Enterohemorrhagic Escherichia coli, Staphylococcus aureus (e.g., methicillin-resistant Staphylococcus aureus), Salmonella, Botulinus, Candida and the like.

In some examples, the respiratory disease can be an inflammatory lung disease or a chronic lung disease having a dysregulated inflammatory process that can be modulated by, for example, nitric oxide. For example, the respiratory disease can be asthma, COPD, chronic bronchitis, bronchiectasis, or cystic fibrosis. In some examples, the respiratory disease can be a lung disease having a cardiovascular component that can be modulated by, for example, nitric oxide. For example, the respiratory disease can be atherosclerosis, post-angioplasty, restenosis, coronary artery diseases or angina. Optionally, the subject can have nontuberculous mycobacterial (NTM) lung disease and/or Lady Windermere syndrome (LWS). In some cases, the subject can be a lung transplant patient.

The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of a respiratory disease), during early onset (e.g., upon initial signs and symptoms of a respiratory disease), or after an established respiratory disease. Prophylactic administration can occur for several hours to years prior to the manifestation of symptoms of an infection. Prophylactic administration can be used, for example, in the preventative treatment of subjects or surfaces exposed to Pseudomonas aeruginosa or to prevent exacerbations. Therapeutic treatment involves contacting the subject with a therapeutically effective amount of the compositions as described herein.

### VI. Stable Compositions and Kits

Also provided herein is a stable composition including a composition as described herein. Optionally, the stable composition described herein includes a nitric oxide (NO) releasing compound, such as a diazeniumdiolate compound, in aqueous conditions. In some examples, the stable composition can include a composition comprising an inert solvent (e.g., water) and a nitric oxide releasing compound comprises at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation, wherein the pH of the composition is maintained in a range of from 5.5 to 8.5 and the osmolality of the composition is from 270 mOsm/kg to 1300 mOsm/kg.

When in the form of composition diluted with an inert solvent, as described herein and further below in the Example, the compositions described herein are appropriately stable to be suitable for formulation preparation and effective administration to a subject. Optionally, the composition is an inhalable composition.

The stable compositions can optionally be provided in the form of a kit. A kit can include any of the compositions described herein. For example, a kit can include a compound of Formula I and a carrier (e.g., a pharmaceutically acceptable carrier).

A kit can include a means for delivery. In some cases, a kit can include a means for delivery by inhalation (e.g., an inhaler, such as a soft mist inhaler, a metered dose inhaler, or a dry powder inhaler; a container attached to a pump; a spray; an electronic vaporizing device; or a nebulizer). A kit can additionally include directions for use of the kit (e.g., instructions for treating a subject or contacting a surface), one or more containers (for the compound(s), composition(s), one or more additives, one or more additional active agents, a means for administering the compounds or compositions, and/or a carrier. Optionally, the stable composition kit can include one or more containers.

As used herein the terms treatment, treat, or treating refer to a method of reducing one or more symptoms of a disease or condition. Thus, in the disclosed method, treatment can refer to a 5%, 10%, 20%, 30%, 40%, 50%, 60%, 70%, 80%, 90%, or 100% reduction in the severity of one or more symptoms of the disease or condition. For example, a method for treating a disease is considered to be a treatment if there is a 5% reduction in one or more symptoms or signs of the disease in a subject as compared to a control. As used herein, control refers to the untreated condition. Thus, the reduction can be a 5%, 10%, 20%, 30%, 40%, 50%, 60%, 70%, 80%, 90%, 100%, or any percent reduction in between 5% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder.

As used herein, references to decreasing, reducing, or inhibiting include a change of 5%, 10%, 20%, 30%, 40%, 50%, 60%, 70%, 80%, 90% or greater as compared to a control level. Such terms can include, but do not necessarily include, complete elimination.

As used herein, subject means both mammals and non-mammals. Mammals include, for example, humans; non-human primates, e.g., apes and monkeys; cattle; horses; sheep; rats; mice; pigs; and goats. Non-mammals include, for example, fish and birds.

Throughout this application, various publications are referenced. The disclosures of these publications in their entireties are hereby incorporated by reference into this application.

### Illustrations

A collection of exemplary embodiments is provided below, including at least some explicitly enumerated as “Illustrations” providing additional description of a variety of example embodiments in accordance with the concepts described herein. These illustrations are not meant to be mutually exclusive, exhaustive, or restrictive; and the disclosure not limited to these example illustrations but rather encompasses all possible modifications and variations within the scope of the issued claims and their equivalents.

Illustration 1. A composition, comprising: a nitric oxide (NO) releasing compound; and an aqueous carrier, wherein the composition exhibits a heat of decomposition of no greater than 300 J/g.

Illustration 2. The composition of any preceding or subsequent illustrations or combination of illustrations, wherein the composition is formulated for inhalation.

Illustration 3. The composition of any preceding or subsequent illustrations or combination of illustrations, wherein the composition further comprises a pharmaceutically acceptable carrier.

Illustration 4. The composition of any preceding or subsequent illustrations or combination of illustrations, wherein the composition further comprises one or more additives.

Illustration 5. The composition of any preceding or subsequent illustrations or combination of illustrations, wherein the one or more additives comprises one or more carbohydrates, organic salts, amino acids, preservatives, salts, chelators, viscosity modifiers, stabilizers, surfactants, antioxidants, polymers, mucolytics, glycols, or cosolvents.

Illustration 6. The composition of any preceding or subsequent illustrations or combination of illustrations, wherein the composition further comprises an additional active agent.

Illustration 7. The composition of any preceding or subsequent illustrations or combination of illustrations, wherein the additional active agent is selected from the group consisting of a peptide, a protein, a corticosteroid, an antibiotic, a short acting beta agonist, a long acting beta agonist, an anti-cholinergic, an antiviral, a monoclonal antibody (mAb), and an antifungal.

Illustration 8. The composition of any preceding or subsequent illustrations or combination of illustrations, wherein the nitric oxide (NO) releasing compound comprises at least one of a diazeniumdiolate, a nitrosothiol, a nitrosourea, an organic nitrate, an organic nitrite, a metal-NO complex, an N-nitroimine, a C-nitroso compound, an oxime, an N-hydroxyguanidine, or a hydroxyurea.

Illustration 9. The composition of any preceding or subsequent illustrations or combination of illustrations, wherein the nitric oxide (NO) releasing compound comprises at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

Illustration 10. The composition of any preceding or subsequent illustrations or combination of illustrations, wherein the compound has the following structure:

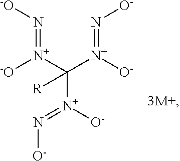

wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H;
a, b, c, d, e, and f are each independently selected from an integer of 0, 1, 2, 3, 4, 5, 6, 7, 8, 9, or 10; and
M₊ is a pharmaceutically-acceptable cation, wherein a ratio of the compound to the cation is such that the overall net charge of the compound is neutral.

Illustration 11. The composition of any preceding or subsequent illustrations or combination of illustrations, wherein the cation is selected from the group consisting of sodium, potassium, lithium, calcium, magnesium, ammonium, and substituted ammonium.

Illustration 12. The composition of any preceding or subsequent illustrations or combination of illustrations, wherein the compound has the following structure:

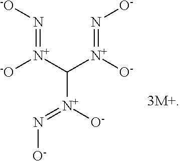

Illustration 13. The composition of any preceding or subsequent illustrations or combination of illustrations, wherein the compound has the following structure:

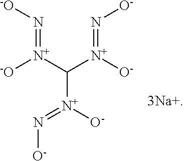

Illustration 14. A method for treating a respiratory disease in a subject, comprising administering to the subject an effective amount of a composition of any preceding illustrations or combination of illustrations.

Illustration 15. The method of any preceding or subsequent illustrations or combination of illustrations, wherein the composition is contained in a drug administration device.

Illustration 16. The method of any preceding or subsequent illustrations or combination of illustrations, wherein the drug administration device comprises a container attached to a pump.

Illustration 17. The method of any preceding or subsequent illustrations or combination of illustrations, wherein the administering comprises aerosolizing the composition.

Illustration 18. The method of any preceding or subsequent illustrations or combination of illustrations, wherein the drug administration device is at least one of a spray and a soft mist inhaler.

Illustration 19. A composition, comprising: a nitric oxide (NO) releasing compound; and a propellant, wherein the composition exhibits a heat of decomposition of no greater than 300 J/g.

Illustration 20. The composition of any preceding or subsequent illustrations or combination of illustrations, wherein the composition is formulated for administration by an inhaler.

Illustration 21. The composition of any preceding or subsequent illustrations or combination of illustrations, wherein the composition is formulated for administration by a metered dose inhaler.

Illustration 22. The composition of any preceding or subsequent illustrations or combination of illustrations, wherein the propellant is a fluorinated hydrocarbon.

Illustration 23. The composition of any preceding or subsequent illustrations or combination of illustrations, wherein the fluorinated hydrocarbon is 1,1,1,2-tetrafluoroethane, 1,1,1,2,3,3,3-heptafluoropropane, trans-1,3,3,3-tetrafluoropropene, 1,1-difluoroethane, or a combination thereof.

Illustration 24. The composition of any preceding or subsequent illustrations or combination of illustrations, wherein the composition further comprises one or more additives.

Illustration 25. The composition of any preceding or subsequent illustrations or combination of illustrations, wherein the one or more additives comprises one or more carbohydrates, organic salts, amino acids, preservatives, salts, chelators, viscosity modifiers, stabilizers, surfactants, antioxidants, polymers, mucolytics, glycols, or cosolvents.

Illustration 26. The composition of any preceding or subsequent illustrations or combination of illustrations, wherein the composition further comprises an additional active agent.

Illustration 27. The composition of any preceding or subsequent illustrations or combination of illustrations, wherein the additional active agent is selected from the group consisting of a peptide, a protein, a corticosteroid, an antibiotic, a short acting beta agonist, a long acting beta agonist, an anti-cholinergic, an antiviral, a monoclonal antibody (mAb), and an antifungal.

Illustration 28. The composition of any preceding or subsequent illustrations or combination of illustrations, wherein the composition further comprises ethanol.

Illustration 29. The composition of any preceding or subsequent illustrations or combination of illustrations, wherein the nitric oxide (NO) releasing compound comprises at least one of a diazeniumdiolate, a nitrosothiol, a nitrosourea, an organic nitrate, an organic nitrite, a metal-NO complex, an N-nitroimine, a C-nitroso compound, an oxime, an N-hydroxyguanidine, or a hydroxyurea.

Illustration 30. The composition of any preceding or subsequent illustrations or combination of illustrations, wherein the nitric oxide (NO) releasing compound comprises at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

Illustration 31. The composition of any preceding or subsequent illustrations or combination of illustrations, wherein the compound has the following structure:

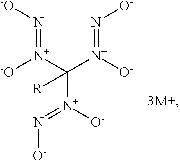

wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H;
a, b, c, d, e, and f are each independently selected from an integer of 0, 1, 2, 3, 4, 5, 6, 7, 8, 9, or 10; and
M₊ is a pharmaceutically-acceptable cation, wherein a ratio of the compound to the cation is such that the overall net charge of the compound is neutral.

Illustration 32. The composition of any preceding or subsequent illustrations or combination of illustrations, wherein the cation is selected from the group consisting of sodium, potassium, lithium, calcium, magnesium, ammonium, and substituted ammonium.

Illustration 33. The composition of any preceding or subsequent illustrations or combination of illustrations, wherein the compound has the following structure:

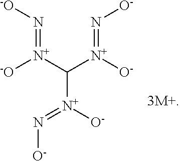

Illustration 34. The composition of any preceding or subsequent illustrations or combination of illustrations, wherein the compound has the following structure:

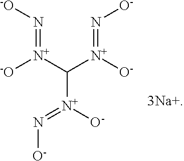

Illustration 35. A method for treating a respiratory disease in a subject, comprising administering to the subject an effective amount of a composition of any preceding illustrations or combination of illustrations.

Illustration 36. The method of any preceding or subsequent illustrations or combination of illustrations, wherein the composition is contained in a drug administration device.

Illustration 37. The method of any preceding or subsequent illustrations or combination of illustrations, wherein the drug administration device is a propellant administration device.

Illustration 38. The method of any preceding or subsequent illustrations or combination of illustrations, wherein the administering comprises aerosolizing the composition.

Illustration 39. The method of any preceding or subsequent illustrations or combination of illustrations, wherein the drug administration device is a metered dose inhaler.

Illustration 40. A composition, comprising: a nitric oxide (NO) releasing compound; and an aqueous based vaporizable liquid, wherein the composition exhibits a heat of decomposition of no greater than 300 J/g.

Illustration 41. The composition of any preceding or subsequent illustrations or combination of illustrations, wherein the aqueous based vaporizable liquid comprises water and one or more of propylene glycol and vegetable glycerine.

Illustration 42. The composition of any preceding or subsequent illustrations or combination of illustrations, wherein the composition is formulated for administration by an electronic vaporizing device.

Illustration 43. The composition of any preceding or subsequent illustrations or combination of illustrations, wherein the composition further comprises one or more additives.

Illustration 44. The composition of any preceding or subsequent illustrations or combination of illustrations, wherein the one or more additives comprises one or more carbohydrates, organic salts, amino acids, preservatives, salts, chelators, viscosity modifiers, stabilizers, surfactants, antioxidants, polymers, mucolytics, glycols, or cosolvents.

Illustration 45. The composition of any preceding or subsequent illustrations or combination of illustrations, wherein the composition further comprises an additional active agent.

Illustration 46. The composition of any preceding or subsequent illustrations or combination of illustrations, wherein the additional active agent is selected from the group consisting of a peptide, a protein, a corticosteroid, an antibiotic, a short acting beta agonist, a long acting beta agonist, an anti-cholinergic, an antiviral, a monoclonal antibody (mAb), and an antifungal.

Illustration 47. The composition of any preceding or subsequent illustrations or combination of illustrations, wherein the nitric oxide (NO) releasing compound comprises at least one of a diazeniumdiolate, a nitrosothiol, a nitrosourea, an organic nitrate, an organic nitrite, a metal-NO complex, an N-nitroimine, a C-nitroso compound, an oxime, an N-hydroxyguanidine, or a hydroxyurea.

Illustration 48. The composition of any preceding or subsequent illustrations or combination of illustrations, wherein the nitric oxide (NO) releasing compound comprises at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

Illustration 49. The composition of any preceding or subsequent illustrations or combination of illustrations, wherein the compound has the following structure:

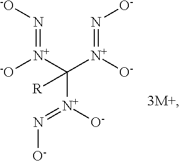

wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H;
a, b, c, d, e, and f are each independently selected from an integer of 0, 1, 2, 3, 4, 5, 6, 7, 8, 9, or 10; and
M₊ is a pharmaceutically-acceptable cation, wherein a ratio of the compound to the cation is such that the overall net charge of the compound is neutral.

Illustration 50. The composition of any preceding or subsequent illustrations or combination of illustrations, wherein the cation is selected from the group consisting of sodium, potassium, lithium, calcium, magnesium, ammonium, and substituted ammonium.

Illustration 51. The composition of any preceding or subsequent illustrations or combination of illustrations, wherein the compound has the following structure:

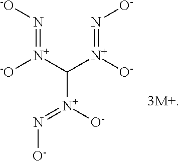

Illustration 52. The composition of any preceding or subsequent illustrations or combination of illustrations, wherein the compound has the following structure:

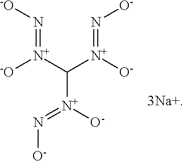

Illustration 53. A method for treating a respiratory disease in a subject, comprising administering to the subject an effective amount of a composition of any preceding illustrations or combination of illustrations.

Illustration 54. The method of any preceding or subsequent illustrations or combination of illustrations, wherein the composition is contained in a drug administration device.

Illustration 55. The method of any preceding or subsequent illustrations or combination of illustrations, wherein the drug administration device is an electronic vaporizing device.

Illustration 56. The method of any preceding or subsequent illustrations or combination of illustrations, wherein the administering comprises vaporizing the composition.

Illustration 57. A composition, comprising: a nitric oxide (NO) releasing compound; and a carrier particle, wherein the composition exhibits a heat of decomposition of no greater than 300 J/g.

Illustration 58. The composition of any preceding or subsequent illustrations or combination of illustrations, wherein the composition is formulated for inhalation.

Illustration 59. The composition of any preceding or subsequent illustrations or combination of illustrations, wherein the carrier particle is at least one of lactose and a stearate treated lactose.

Illustration 60. The composition of any preceding or subsequent illustrations or combination of illustrations, wherein the stearate treated lactose comprises a metal cationic salt selected from the group consisting of a magnesium salt, a potassium salt, a sodium salt, or a calcium salt.

Illustration 61. The composition of any preceding or subsequent illustrations or combination of illustrations, wherein the composition further comprises one or more additives.

Illustration 62. The composition of any preceding or subsequent illustrations or combination of illustrations, wherein the one or more additives comprises one or more carbohydrates, organic salts, amino acids, preservatives, salts, chelators, viscosity modifiers, stabilizers, surfactants, antioxidants, polymers, mucolytics, glycols, or cosolvents.

Illustration 63. The composition of any preceding or subsequent illustrations or combination of illustrations, wherein the composition further comprises an additional active agent.

Illustration 64. The composition of any preceding or subsequent illustrations or combination of illustrations, wherein the additional active agent is selected from the group consisting of a peptide, a protein, a corticosteroid, an antibiotic, a short acting beta agonist, a long acting beta agonist, an anti-cholinergic, an antiviral, a monoclonal antibody (mAb), and an antifungal.

Illustration 65. The composition of any preceding or subsequent illustrations or combination of illustrations, wherein the nitric oxide (NO) releasing compound comprises at least one of a diazeniumdiolate, a nitrosothiol, a nitrosourea, an organic nitrate, an organic nitrite, a metal-NO complex, an N-nitroimine, a C-nitroso compound, an oxime, an N-hydroxyguanidine, or a hydroxyurea.

Illustration 66. The composition of any preceding or subsequent illustrations or combination of illustrations, wherein the nitric oxide (NO) releasing compound comprises at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

Illustration 67. The composition of any preceding or subsequent illustrations or combination of illustrations, wherein the compound has the following structure:

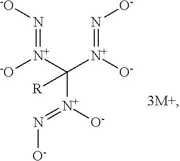

wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H;
a, b, c, d, e, and f are each independently selected from an integer of 0, 1, 2, 3, 4, 5, 6, 7, 8, 9, or 10; and
M₊ is a pharmaceutically-acceptable cation, wherein a ratio of the compound to the cation is such that the overall net charge of the compound is neutral.

Illustration 68. The composition of any preceding or subsequent illustrations or combination of illustrations, wherein the cation is selected from the group consisting of sodium, potassium, lithium, calcium, magnesium, ammonium, and substituted ammonium.

Illustration 69. The composition of any preceding or subsequent illustrations or combination of illustrations, wherein the compound has the following structure:

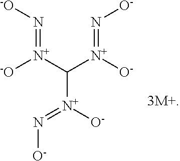

Illustration 70. The composition of any preceding or subsequent illustrations or combination of illustrations, wherein the compound has the following structure:

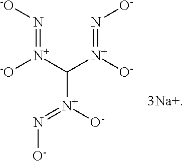

Illustration 71. A method for treating a respiratory disease in a subject, comprising administering to the subject an effective amount of a composition of any preceding illustrations or combination of illustrations.

Illustration 72. The method of any preceding or subsequent illustrations or combination of illustrations, wherein the composition is contained in a drug administration device.

Illustration 73. The method of any preceding or subsequent illustrations or combination of illustrations, wherein the drug administration device is a dry powder inhaler.

Illustration 74. The method of any preceding or subsequent illustrations or combination of illustrations, wherein the administering comprises aerosolizing the composition.

Illustration 75. A composition, comprising: a porous particle comprising a nitric oxide (NO) releasing compound, wherein the composition exhibits a heat of decomposition of no greater than 300 J/g.

Illustration 76. The composition of any preceding or subsequent illustrations or combination of illustrations, wherein the composition is formulated for administration by an inhaler.

Illustration 77. The composition of any preceding or subsequent illustrations or combination of illustrations, wherein the composition is formulated for administration by a dry powder inhaler.

Illustration 78. The composition of any preceding or subsequent illustrations or combination of illustrations, wherein a mass median aerodynamic diameter of the porous particle is no greater than 10 microns in diameter.

Illustration 79. The composition of any preceding or subsequent illustrations or combination of illustrations, wherein the mass median aerodynamic diameter of the porous particle is no greater than 5 microns in diameter.

Illustration 80. The composition of any preceding or subsequent illustrations or combination of illustrations, wherein the mass median aerodynamic diameter of the porous particle is no greater than 3 microns in diameter.

Illustration 81. The composition of any preceding or subsequent illustrations or combination of illustrations, wherein the composition further comprises one or more additives.

Illustration 82. The composition of any preceding or subsequent illustrations or combination of illustrations, wherein the one or more additives comprises one or more carbohydrates, organic salts, amino acids, preservatives, salts, chelators, viscosity modifiers, stabilizers, surfactants, antioxidants, polymers, mucolytics, glycols, or cosolvents.

Illustration 83. The composition of any preceding or subsequent illustrations or combination of illustrations, wherein the composition further comprises an additional active agent.

Illustration 84. The composition of any preceding or subsequent illustrations or combination of illustrations, wherein the additional active agent is selected from the group consisting of a peptide, a protein, a corticosteroid, an antibiotic, a short acting beta agonist, a long acting beta agonist, an anti-cholinergic, an antiviral, a monoclonal antibody (mAb), and an antifungal.

Illustration 85. The composition of any preceding or subsequent illustrations or combination of illustrations, wherein the nitric oxide (NO) releasing compound comprises at least one of a diazeniumdiolate, a nitrosothiol, a nitrosourea, an organic nitrate, an organic nitrite, a metal-NO complex, an N-nitroimine, a C-nitroso compound, an oxime, an N-hydroxyguanidine, or a hydroxyurea.

Illustration 86. The composition of any preceding or subsequent illustrations or combination of illustrations, wherein the nitric oxide (NO) releasing compound comprises at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

Illustration 87. The composition of any preceding or subsequent illustrations or combination of illustrations, wherein the compound has the following structure:

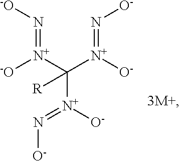

wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H;
a, b, c, d, e, and f are each independently selected from an integer of 0, 1, 2, 3, 4, 5, 6, 7, 8, 9, or 10; and
M₊ is a pharmaceutically-acceptable cation, wherein a ratio of the compound to the cation is such that the overall net charge of the compound is neutral.

Illustration 88. The composition of any preceding or subsequent illustrations or combination of illustrations, wherein the cation is selected from the group consisting of sodium, potassium, lithium, calcium, magnesium, ammonium, and substituted ammonium.

Illustration 89. The composition of any preceding or subsequent illustrations or combination of illustrations, wherein the compound has the following structure:

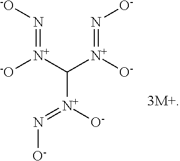

Illustration 90. The composition of any preceding or subsequent illustrations or combination of illustrations, wherein the compound has the following structure:

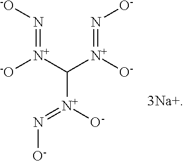

Illustration 91. A method for treating a respiratory disease in a subject, comprising administering to the subject an effective amount of a composition of any preceding illustrations or combination of illustrations.

Illustration 92. The method of any preceding or subsequent illustrations or combination of illustrations, wherein the composition is contained in a drug administration device.

Illustration 93. The method of any preceding or subsequent illustrations or combination of illustrations, wherein the drug administration device is a dry powder inhaler.

Illustration 94. A pharmaceutical composition formulated for inhalation, comprising: a nitric oxide (NO) releasing compound; and a pharmaceutically-acceptable carrier, wherein the pharmaceutical composition exhibits a heat of decomposition of no greater than 300 J/g, and is administered via a drug administration device.

Illustration 95. The pharmaceutical composition of any preceding or subsequent illustrations or combination of illustrations, wherein the pharmaceutical composition further comprises one or more additives.

Illustration 96. The pharmaceutical composition of any preceding or subsequent illustrations or combination of illustrations, wherein the one or more additives comprises one or more carbohydrates, organic salts, amino acids, preservatives, salts, chelators, viscosity modifiers, stabilizers, surfactants, antioxidants, polymers, mucolytics, glycols, or cosolvents.

Illustration 97. The pharmaceutical composition of any preceding or subsequent illustrations or combination of illustrations, wherein the pharmaceutical composition further comprises an additional active agent.

Illustration 98. The pharmaceutical composition of any preceding or subsequent illustrations or combination of illustrations, wherein the additional active agent is selected from the group consisting of a peptide, a protein, a corticosteroid, an antibiotic, a short acting beta agonist, a long acting beta agonist, an anti-cholinergic, an antiviral, a monoclonal antibody (mAb), and an antifungal.

Illustration 99. The pharmaceutical composition of any preceding or subsequent illustrations or combination of illustrations, wherein the nitric oxide (NO) releasing compound comprises at least one of a N-diazeniumdiolate, a C-diazeniumdiolate, a nitrosothiol, a nitrosourea, an organic nitrate, an organic nitrite, a metal-NO complex, an N-nitroimine, a C-nitroso compound, an oxime, an N-hydroxyguanidine, or a hydroxyurea.

Illustration 100. The pharmaceutical composition of any preceding or subsequent illustrations or combination of illustrations, wherein the nitric oxide (NO) releasing compound comprises at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

Illustration 101. The pharmaceutical composition of any preceding or subsequent illustrations or combination of illustrations, wherein the compound has the following structure:

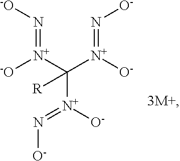

wherein;
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H;
a, b, c, d, e, and f are each independently selected from an integer of 0, 1, 2, 3, 4, 5, 6, 7, 8, 9, or 10; and
M₊ is a pharmaceutically-acceptable cation, wherein a ratio of the compound to the cation is such that the overall net charge of the compound is neutral.

Illustration 102. The pharmaceutical composition of any preceding or subsequent illustrations or combination of illustrations, wherein the cation is selected from the group consisting of sodium, potassium, lithium, calcium, magnesium, ammonium, and substituted ammonium.

Illustration 103. The pharmaceutical composition of any preceding or subsequent illustrations or combination of illustrations, wherein the compound has the following structure:

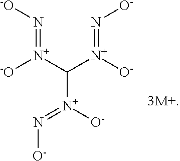

Illustration 104. The pharmaceutical composition of any preceding or subsequent illustrations or combination of illustrations, wherein the compound has the following structure:

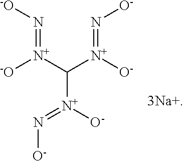

Illustration 105. The pharmaceutical composition of any preceding or subsequent illustrations or combination of illustrations, wherein the pharmaceutically-acceptable carrier is an aqueous carrier.

Illustration 106. The pharmaceutical composition of any preceding or subsequent illustrations or combination of illustrations, wherein the pharmaceutically-acceptable carrier is a propellant.

Illustration 107. The pharmaceutical composition of any preceding or subsequent illustrations or combination of illustrations, wherein the propellant is a fluorinated hydrocarbon.

Illustration 108. The pharmaceutical composition of any preceding or subsequent illustrations or combination of illustrations, wherein the fluorinated hydrocarbon is 1,1,1,2-tetrafluoroethane, 1,1,1,2,3,3,3-heptafluoropropane, trans-1,3,3,3-tetrafluoropropene, 1,1-difluoroethane, or a combination thereof.

Illustration 109. The pharmaceutical composition of any preceding or subsequent illustrations or combination of illustrations, wherein the pharmaceutical composition further comprises ethanol.

Illustration 110. The pharmaceutical composition of any preceding or subsequent illustrations or combination of illustrations, wherein the pharmaceutically-acceptable carrier is an aqueous based vaporizable liquid.

Illustration 111. The pharmaceutical composition of any preceding or subsequent illustrations or combination of illustrations, wherein the aqueous based vaporizable liquid comprises water and one or more of propylene glycol and vegetable glycerine.

Illustration 112. A method for treating a respiratory disease in a subject, comprising administering to the subject an effective amount of a pharmaceutical composition of any preceding illustrations or combination of illustrations.

Illustration 113. The method of any preceding or subsequent illustrations or combination of illustrations, wherein the pharmaceutical composition is contained in a drug administration device.

Illustration 114. The method of any preceding or subsequent illustrations or combination of illustrations, wherein the drug administration device is a container attached to a pump, a spray, a soft mist inhaler, a metered dose inhaler, a propellant administration device, or an electronic vaporizing device.

Illustration 115. The method of any preceding or subsequent illustrations or combination of illustrations, wherein the administering comprises aerosolizing the pharmaceutical composition or vaporizing the pharmaceutical composition.

Illustration 116. A pharmaceutical composition formulated for administration by a dry powder inhaler, comprising: a nitric oxide (NO) releasing compound; and a pharmaceutically acceptable carrier particle, wherein a mass median aerodynamic diameter of the pharmaceutically acceptable carrier particle is no greater than 10 microns in diameter, wherein the pharmaceutical composition exhibits a heat of decomposition of no greater than 300 J/g.

Illustration 117. The pharmaceutical composition of any preceding or subsequent illustrations or combination of illustrations, wherein the pharmaceutically acceptable carrier particle is a solid particle or a porous particle.

Illustration 118. The pharmaceutical composition of any preceding or subsequent illustrations or combination of illustrations, wherein the pharmaceutically acceptable carrier particle comprises at least one of lactose and a stearate treated lactose.

Illustration 119. The pharmaceutical composition of any preceding or subsequent illustrations or combination of illustrations, wherein the stearate treated lactose comprises a metal cationic salt selected from the group consisting of a magnesium salt, a potassium salt, a sodium salt, or a calcium salt.

Illustration 120. The pharmaceutical composition of any preceding or subsequent illustrations or combination of illustrations, wherein the mass median aerodynamic diameter of the pharmaceutically acceptable carrier particle is no greater than 5 microns in diameter.

Illustration 121. The pharmaceutical composition of any preceding or subsequent illustrations or combination of illustrations, wherein the mass median aerodynamic diameter of the pharmaceutically acceptable carrier particle is no greater than 3 microns in diameter.

Illustration 122. The pharmaceutical composition of any preceding or subsequent illustrations or combination of illustrations, wherein the pharmaceutical composition further comprises one or more additives.

Illustration 123. The pharmaceutical composition of any preceding or subsequent illustrations or combination of illustrations, wherein the one or more additives comprises one or more carbohydrates, organic salts, amino acids, preservatives, salts, chelators, viscosity modifiers, stabilizers, surfactants, antioxidants, polymers, mucolytics, glycols, or cosolvents.

Illustration 124. The pharmaceutical composition of any preceding or subsequent illustrations or combination of illustrations, wherein the pharmaceutical composition further comprises an additional active agent.

Illustration 125. The pharmaceutical composition of any preceding or subsequent illustrations or combination of illustrations, wherein the additional active agent is selected from the group consisting of a peptide, a protein, a corticosteroid, an antibiotic, a short acting beta agonist, a long-acting beta agonist, an anti-cholinergic, an antiviral, a monoclonal antibody (mAb), and an antifungal.

Illustration 126. The pharmaceutical composition of any preceding or subsequent illustrations or combination of illustrations, wherein the nitric oxide (NO) releasing compound comprises at least one of a N-diazeniumdiolate, a C-diazeniumdiolate, a nitrosothiol, a nitrosourea, an organic nitrate, an organic nitrite, a metal-NO complex, an N-nitroimine, a C-nitroso compound, an oxime, an N-hydroxyguanidine, or a hydroxyurea.

Illustration 127. The pharmaceutical composition of any preceding or subsequent illustrations or combination of illustrations, wherein the nitric oxide (NO) releasing compound comprises at least two diazeniumdiolate groups on one carbon atom, each having a charge and each with an associated pharmaceutically acceptable cation to balance the charge on the diazeniumdiolate groups, which compound has a molecular weight below 500 g/mol, not including the associated pharmaceutically acceptable cation.

Illustration 128. The pharmaceutical composition of any preceding or subsequent illustrations or combination of illustrations, wherein the compound has the following structure:

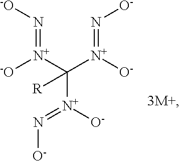

wherein;
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H;
a, b, c, d, e, and f are each independently selected from an integer of 0, 1, 2, 3, 4, 5, 6, 7, 8, 9, or 10; and
M₊ is a pharmaceutically-acceptable cation, wherein a ratio of the compound to the cation is such that the overall net charge of the compound is neutral.

Illustration 129. The pharmaceutical composition of any preceding or subsequent illustrations or combination of illustrations, wherein the cation is selected from the group consisting of sodium, potassium, lithium, calcium, magnesium, ammonium, and substituted ammonium.

Illustration 130. The pharmaceutical composition of any preceding or subsequent illustrations or combination of illustrations, wherein the compound has the following structure:

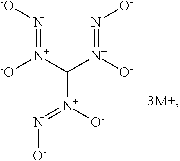

Illustration 131. A method for treating a respiratory disease in a subject, comprising administering to the subject an effective amount of a pharmaceutical composition of any of the preceding illustrations or combination of illustrations.

Illustration 132. The method for treating a respiratory disease in a subject of any preceding or subsequent illustrations or combination of illustrations, wherein the pharmaceutical composition is contained in a drug administration device.

Illustration 133. The method for treating a respiratory disease in a subject of any preceding or subsequent illustrations or combination of illustrations, wherein the drug administration device is a dry powder inhaler.

Illustration 134. The method for treating a respiratory disease in a subject of any preceding or subsequent illustrations or combination of illustrations, wherein the administering comprises aerosolizing the pharmaceutical composition.

### EXAMPLE

The following example is set forth below to illustrate the methods and results according to the disclosed subject matter. This example is not intended to be inclusive of all aspects of the subject matter disclosed herein, but rather to illustrate representative methods and results. This example is not intended to exclude equivalents and variations of the subject matter described herein which are apparent to one skilled in the art.

### Example 1: Thermal Decomposition Properties

Given MD3's chemical structure and potential for exothermic decomposition, experiments were designed and performed to fully evaluate its thermal decomposition properties and impact sensitivity. Specifically, the thermal decomposition properties of MD3 were characterized using differential scanning calorimetry. As expected, MD3 undergoes violent exothermic decomposition (detonation) in the solid state at 187° C. Likewise, the decomposition energy was determined to be 3754 J/g. As background, a substance is considered a candidate for classification as a United Nations Class 1 explosive if the heat of decomposition is ≥500 J/g. While a substance with a higher energy content is not necessarily highly sensitive or hazardous, the sensitivity of a high energy content material must be understood to ensure safe handling. Therefore, MD3's sensitivity to impact, friction forces, and electrostatic discharge was tested.

The threshold initiation limit (TIL₆) is the lowest energy level that does not cause the material to detonate when the test is replicated six times. The TIL₆ for solid MD3 was 7.5 J of impact energy. For reference, highly sensitive materials will decompose/detonate at energies of 2 J or less. Most importantly, wetting MD3 with water at a 1 to 1 ratio resulted in no decomposition up to 50 J of impact energy. Likewise, the TIL₆ for solid MD3 was 80 N of frictional force. When wetted with water at a 1 to 1 ratio, no decomposition of MD3 was observed up to 360 N. Finally, the TIL₆ for solid MD3 was 63 mJ of electrostatic discharge (at 5 KV). When wetted with water at a 1 to 1 weight ratio, no decomposition of MD3 was observed up to 4.5 J (at 30 KV).

As MD3 was effectively desensitized to impact, frictional forces, and electrostatic discharge by dilution in an inert substance (water), control procedures to process/store/handle/ship MD3 as an aqueous solution were designed and developed. By blending with higher ratios of water, the energy content of the MD3 mixture/solution was diluted to the point where the mixture is no longer classified as an explosive or self-reactive material. For example, at a concentration of 64 mg/mL, the energy content of the solution remains below 300 J/g. As the final form of the MD3 drug substance is a solution, special control of the solid form is not required.

The compositions and methods of the appended claims are not limited in scope by the specific compositions and methods described herein, which are intended as illustrations of a few aspects of the claims and any compositions and methods that are functionally equivalent are within the scope of this disclosure. Various modifications of the compositions and methods in addition to those shown and described herein are intended to fall within the scope of the appended claims. Further, while only certain representative compositions, methods, and aspects of these compositions and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

What is claimed is:

1. A pharmaceutical composition formulated for administration by a dry powder inhaler, comprising:
a nitric oxide (NO) releasing compound; and
a pharmaceutically acceptable carrier particle, wherein a mass median aerodynamic diameter of the pharmaceutically acceptable carrier particle is no greater than 10 microns in diameter,
wherein the pharmaceutical composition exhibits a heat of decomposition of no greater than 300 J/g.

2. The pharmaceutical composition of claim 1, wherein the pharmaceutically acceptable carrier particle is a solid particle or a porous particle.

3. The pharmaceutical composition of claim 1, wherein the pharmaceutically acceptable carrier particle comprises at least one of lactose and a stearate treated lactose.

4. The pharmaceutical composition of claim 3, wherein the stearate treated lactose comprises a metal cationic salt selected from the group consisting of a magnesium salt, a potassium salt, a sodium salt, or a calcium salt.

5. The pharmaceutical composition of claim 1, wherein the mass median aerodynamic diameter of the pharmaceutically acceptable carrier particle is no greater than 5 microns in diameter.

6. The pharmaceutical composition of claim 1, wherein the mass median aerodynamic diameter of the pharmaceutically acceptable carrier particle is no greater than 3 microns in diameter.

7. The pharmaceutical composition of claim 1, wherein the pharmaceutical composition further comprises one or more additives.

8. The pharmaceutical composition of claim 7, wherein the one or more additives comprises one or more carbohydrates, organic salts, amino acids, preservatives, salts, chelators, viscosity modifiers, stabilizers, surfactants, antioxidants, polymers, mucolytics, glycols, or cosolvents.

9. The pharmaceutical composition of claim 1, wherein the pharmaceutical composition further comprises an additional active agent.

10. The pharmaceutical composition of claim 9, wherein the additional active agent is selected from the group consisting of a peptide, a protein, a corticosteroid, an antibiotic, a short acting beta agonist, a long-acting beta agonist, an anti-cholinergic, an antiviral, a monoclonal antibody (mAb), and an antifungal.

11. The pharmaceutical composition of claim 1, wherein the nitric oxide (NO) releasing compound comprises at least one of a diazeniumdiolate, a nitrosothiol, a nitrosourea, an organic nitrate, an organic nitrite, a metal-NO complex, an N-nitroimine, a C-nitroso compound, an oxime, an N-hydroxyguanidine, or a hydroxyurea.

12. The pharmaceutical composition of claim 1, wherein the nitric oxide (NO) releasing compound comprises at least two diazeniumdiolate groups on one carbon atom, each having a charge and each with an associated pharmaceutically acceptable cation to balance the charge on the diazeniumdiolate groups, which compound has a molecular weight below 500 g/mol, not including the associated pharmaceutically acceptable cation.

13. The pharmaceutical composition of claim 12, wherein the compound has the following structure: 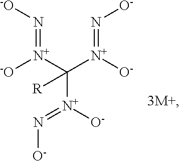
wherein;
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H;
a, b, c, d, e, and f are each independently selected from an integer of 0, 1, 2, 3, 4, 5, 6, 7, 8, 9, or 10; and
M₊ is a pharmaceutically-acceptable cation, wherein a ratio of the compound to the cation is such that the overall net charge of the compound is neutral.

14. The pharmaceutical composition of claim 13, wherein the cation is selected from the group consisting of sodium, potassium, lithium, calcium, magnesium, ammonium, and substituted ammonium.

15. The pharmaceutical composition of claim 13, wherein the compound has the following structure:

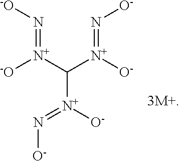

16. A method for treating a respiratory disease in a subject, comprising administering to the subject an effective amount of a pharmaceutical composition of claim 1.

17. The method of claim 16, wherein the pharmaceutical composition is contained in a drug administration device.

18. The method of claim 17, wherein the drug administration device is a dry powder inhaler.

19. The method of claim 16, wherein the administering comprises aerosolizing the pharmaceutical composition.
